(19) United States
(12) Patent Application Publication
(10) Pub. No.: US 2025/0295366 A1
(43) Pub. Date: Sep. 25, 2025

(54) MONITORING OF PHYSIOLOGICAL PARAMETERS WITH WEARABLE DEVICE

(71) Applicant: Masimo Corporation, Irvine, CA (US)

(72) Inventors: Ammar Al-Ali, San Juan Capistrano, CA (US); Konstantinos Michalopoulos, Irvine, CA (US); Jerome J. Novak, JR., Lake Forest, CA (US)

(21) Appl. No.: 19/087,193

(22) Filed: Mar. 21, 2025

Related U.S. Application Data
(60) Provisional application No. 63/568,684, filed on Mar. 22, 2024.

(51) Int. Cl. A61B 5/00; A61B 5/0205; A61B 5/024; A61B 5/08; A61B 5/1455

(52) U.S. Cl. CPC A61B 5/7282; A61B 5/0205; A61B 5/02416; A61B 5/14551; A61B 5/681; A61B 5/7264; A61B 5/746; A61B 5/0826

(57) ABSTRACT

A system is disclosed for continuous and intermittent monitoring of physiological parameters. The system comprises at least one sensor coupled to a wearable device configured to measure physiological parameters of a user; a non-transitory data store storing data collected from the at least one sensor and computer-executable instructions; and a processor in communication with the at least one sensor and the non-transitory data store, wherein the computer-executable instructions, when executed by the processor, configure the processor to: continuously measure the physiological parameters by sampling the at least one sensor at a sampling rate to obtain repeated measurements at series of intervals configured to capture important physiological events; compare measurements of the physiological parameters to a threshold to determine a likelihood of a physiological event; and alert the user when an occurrence of the physiological event is likely.

### INCORPORATION BY REFERENCE TO ANY PRIORITY APPLICATIONS

The present application claims priority benefit to U.S. Provisional Application No. 63/568,684 filed Mar. 22, 2024, entitled “MONITORING OF PHYSIOLOGICAL PARAMETERS WITH WEARABLE DEVICE,” which is hereby incorporated by reference herein in its entirety. Any and all applications for which a foreign or domestic priority claim is identified in the Application Data Sheet as filed with the present application are hereby incorporated by reference under 37 CFR 1.57 and made a part of this specification.

### FIELD

The present disclosure relates to a wearable health monitoring device incorporating a plurality of sensors worn on the wrist.

### BACKGROUND

Continuously monitoring a user's physiological parameters may assist in identifying physiological events for diagnosing health conditions, such as sleep apnea. Continuous monitoring may include pulse oximetry or plethysmography, which utilizes a noninvasive sensor to measure oxygen saturation and pulse rate (PR), among other physiological parameters. Pulse oximetry or plethysmography relies on a sensor attached externally to the patient (typically for example, at the fingertip, foot, ear, forehead, or other measurement sites) to output signals indicative of various physiological parameters, such as a patient's blood constituents and/or analytes, including for example a percent value for arterial oxygen saturation, among other physiological parameters. The sensor has at least one emitter that transmits optical radiation of one or more wavelengths into a tissue site and at least one detector that responds to the intensity of the optical radiation (which can be reflected from or transmitted through the tissue site, such as a surface of a user) after absorption by pulsatile arterial blood flowing within the tissue site. Based upon this response, a processor determines the relative concentrations of oxygenated hemoglobin (HbO2) and deoxygenated hemoglobin (Hb) in the blood so as to derive oxygen saturation, which can provide early detection of potentially hazardous decreases in a patient's oxygen supply, and other physiological parameters.

A patient monitoring device can include a plethysmograph sensor. The plethysmograph sensor can calculate oxygen saturation (SpO2), PR, a plethysmograph waveform, perfusion index (PI), pleth variability index (PVI), methemoglobin (MetHb), carboxyhemoglobin (CoHb), total hemoglobin (tHb), respiration rate, glucose, and/or otherwise. The parameters measured by the plethysmograph sensor can display on one or more monitors the foregoing parameters individually, in groups, in trends, as combinations, or as an overall wellness or other index.

A pulse oximetry sensor is described in U.S. Pat. No. 6,088,607 entitled Low Noise Optical Probe; pulse oximetry signal processing is described in U.S. Pat. Nos. 6,650,917 and 6,699,194 entitled Signal Processing Apparatus and Signal Processing Apparatus and Method, respectively; a pulse oximeter monitor is described in U.S. Pat. No. 6,584,336 entitled Universal/Upgrading Pulse Oximeter; all of which are assigned to Masimo Corporation, Irvine, CA, and each is incorporated by reference herein in its entirety.

### SUMMARY

In some aspects, the techniques described herein relate to a system, including: a battery providing operational power; at least one sensor coupled to a wearable device configured to measure physiological parameters of a user; a non-transitory data store storing data collected from the at least one sensor and computer-executable instructions; and a processor in communication with the at least one sensor and the non-transitory data store, wherein the computer-executable instructions, when executed by the processor, configure the processor to: continuously measure the physiological parameters by sampling the at least one sensor at a sampling rate to obtain repeated measurements at series of intervals configured to capture important physiological events; compare measurements of the physiological parameters to a threshold to determine a likelihood of a physiological event; and alert the user when an occurrence of the physiological event is likely.

In some aspects, the techniques described herein relate to a system, wherein the likelihood of the physiological event is determined in part by detecting deviations of the physiological parameters from at least one baseline and obtaining a confidence score of the physiological event from a correlation between one or more of the physiological parameters.

In some aspects, the techniques described herein relate to a system, wherein further computer-executable instructions, when executed by the processor, configure the processor to alert the user when the likelihood of the physiological event is above a predetermined threshold.

In some aspects, the techniques described herein relate to a system 1-3, wherein the physiological event is likely when the measurements indicate at least one of the physiological parameters is outside of a parameter threshold, wherein the parameter threshold includes a low threshold and a high threshold.

In some aspects, the techniques described herein relate to a system, wherein the low threshold corresponds to at least one of a user baseline or an objective standard.

In some aspects, the techniques described herein relate to a system, wherein the high threshold corresponds to at least one of a user baseline or an objective standard.

In some aspects, the techniques described herein relate to a system, wherein the physiological parameters include at least one of blood oxygen, pulse rate (PR), heart rate (HR), skin temperature, core temperature, ambient temperature, movement, body position, respiration rate, heart rate variability (HRV), pulse rate variability (PRV), electrocardiogram (ECG), or acoustic data.

In some aspects, the techniques described herein relate to a system, wherein the at least one sensor includes at least one of: an optical physiological sensor configured to measure the physiological parameters by receiving reflected light emitted by a wearable device from a surface of a user, a microphone configured to record the physiological parameters of the user corresponding to respiration sounds of the user while sleeping, a temperature sensor configured to measure temperature of the user and/or environment surrounding the user, or an accelerometer configured to measure the physiological parameters used to detect a body position of the user while the user is sleeping.

In some aspects, the techniques described herein relate to a system, wherein the physiological event is a respiratory event.

In some aspects, the techniques described herein relate to a system, wherein the respiratory event includes one or more of respiratory depression, a respiratory obstruction, or a cessation of breathing.

In some aspects, the techniques described herein relate to a system, wherein further computer-executable instructions, when executed by the processor, configure the processor to: continuously measure blood oxygen data and PR data by sampling the at least one sensor at a sampling rate to obtain repeated measurements at series of intervals configured to capture important physiological events.

In some aspects, the techniques described herein relate to a system, wherein the sampling rate corresponds to a data freshness standard.

In some aspects, the techniques described herein relate to a system, wherein further computer-executable instructions, when executed by the processor, configure the processor to: compare the measurements, including blood oxygen data and PR data, to a threshold to determine a likelihood of a respiratory event corresponding to a respiratory depression, a respiratory obstruction, or a cessation of breathing, wherein the likelihood of the respiratory event is determined in part by detecting deviations of the blood oxygen data and the PR data from baselines and obtaining a confidence score of the respiratory event from a correlation between the blood oxygen data and the PR data.

In some aspects, the techniques described herein relate to a system 1-13, wherein the likelihood of the respiratory event is above the predetermined threshold when the confidence score indicates a correlation between a drop in the blood oxygen data and an increase in the PR data.

In some aspects, the techniques described herein relate to a system 1-14, wherein the likelihood of the respiratory event is above the predetermined threshold when the blood oxygen data is outside of blood oxygen thresholds, including a low blood oxygen threshold and a high blood oxygen threshold.

In some aspects, the techniques described herein relate to a system, wherein the low blood oxygen threshold corresponds to at least one of a user baseline or an objective standard.

In some aspects, the techniques described herein relate to a system, wherein the high blood oxygen threshold corresponds to at least one of a user baseline or an objective standard.

In some aspects, the techniques described herein relate to a system 1-14, wherein the likelihood of the respiratory event is above the predetermined threshold when the PR data is outside of PR thresholds, including a low PR threshold and a high PR threshold.

In some aspects, the techniques described herein relate to a system, wherein the low PR threshold corresponds to at least one of a user baseline or an objective standard.

In some aspects, the techniques described herein relate to a system, wherein the high PR threshold corresponds to at least one of a user baseline or an objective standard.

In some aspects, the techniques described herein relate to a system, including: a battery providing operational power; at least one sensor coupled to a wearable device configured to measure physiological parameters of a user; a non-transitory data store storing data from the at least one sensor and computer-executable instructions; and a processor in communication with the at least one sensor and the non-transitory data store, wherein the computer-executable instructions, when executed by the processor, configure the processor to: periodically measure the physiological parameters by sampling the at least one sensor at a sampling rate; compare measurements of the physiological parameters to a threshold determine a likelihood of a physiological event; and update the sampling rate of the at least one sensor when the physiological event is likely.

In some aspects, the techniques described herein relate to a system, wherein the likelihood of the physiological event is determined in part by detecting deviations of the physiological parameters from at least one baseline and obtaining a confidence score of the physiological event from a correlation between one or more of the physiological parameters.

In some aspects, the techniques described herein relate to a system, wherein the physiological event is likely when the measurements indicate at least one of the physiological parameters is outside of a parameter threshold, including a low threshold and a high threshold.

In some aspects, the techniques described herein relate to a system, wherein the low threshold corresponds to at least one of a user baseline or an objective standard.

In some aspects, the techniques described herein relate to a system, wherein the high threshold corresponds to at least one of a user baseline or an objective standard.

In some aspects, the techniques described herein relate to a system 21-25, wherein the physiological parameters include at least one of blood oxygen, pulse rate (PR), heart rate (HR), skin temperature, core temperature, ambient temperature, movement, body position, respiration rate, heart rate variability (HRV), pulse rate variability (PRV), electrocardiogram (ECG), or acoustic data.

In some aspects, the techniques described herein relate to a system 21-26, wherein the at least one sensor includes at least one of: an optical physiological sensor configured to measure the physiological parameters by receiving reflected light emitted by a wearable device from a surface of a user, a microphone configured to record the physiological parameters of the user corresponding to respiration sounds of the user while sleeping, a temperature sensor configured to measure temperature of the user and/or environment surrounding the user, or an accelerometer configured to measure the physiological parameters used to detect a body position of the user while the user is sleeping.

In some aspects, the techniques described herein relate to a system 21-27, wherein the physiological event is a respiratory event.

In some aspects, the techniques described herein relate to a system, wherein the respiratory event includes at least one of respiratory depression, a respiratory obstruction, or a cessation of breathing.

In some aspects, the techniques described herein relate to a system 21-29, wherein further computer-executable instructions, when executed by the processor, configure the processor to: measure body position data by sampling the accelerometer at a second sampling rate.

In some aspects, the techniques described herein relate to a system, wherein further computer-executable instructions, when executed by the processor, configure the processor to: compare the measurements, including body position data of the user, to blood oxygen data to determine a likelihood of a respiratory event corresponding to a respiratory depression, a respiratory obstruction, or a cessation of breathing, wherein the likelihood of the respiratory event is determined in part by detecting a deviation of the blood oxygen data from a baseline and obtaining a confidence score of the respiratory event from a correlation between the blood oxygen data and the body position data.

In some aspects, the techniques described herein relate to a system, wherein further computer-executable instructions, when executed by the processor, configure the processor to: update the sampling rate of the at least one sensor to a new sampling rate to measure blood oxygen data when the likelihood of a respiratory event is above a predetermined threshold, wherein the new sampling rate is greater than the sampling rate.

In some aspects, the techniques described herein relate to a system 21-32, wherein the likelihood of the respiratory event is above the predetermined threshold when the confidence score indicates a correlation between a drop in the blood oxygen data and the body position data of the user in a position increasing a risk of the respiratory event.

In some aspects, the techniques described herein relate to a system 21-32, wherein the likelihood of the respiratory event is above the predetermined threshold when the blood oxygen data is outside of blood oxygen thresholds, including a low blood oxygen threshold and a high blood oxygen threshold.

In some aspects, the techniques described herein relate to a system, wherein the low blood oxygen threshold corresponds to at least one of a user baseline or an objective standard.

In some aspects, the techniques described herein relate to a system, wherein the high blood oxygen threshold corresponds to at least one of a user baseline or an objective standard.

In some aspects, the techniques described herein relate to a method of operating a wearable system for continuously monitoring physiological data of a wearer over an extended period using a set of physiological sensors by dynamically scheduling power consumption of the set of physiological sensors based at least in part on the physiological data, the method including: determining a movement condition of the wearable system based at least in part on data from at least one motion sensor, the movement condition including elevated movement or reduced movement; determining a physiological stability of the wearer based at least in part on at least one physiological signal, the physiological stability including a stable condition or an unstable condition; selecting a mode of operating a plurality of LEDs based at least in part on the movement condition and the physiological stability of the wearer, wherein a first mode of operation is associated with elevated movement or unstable physiological condition, wherein the first mode of operation includes a first power management condition including permitting the plurality of LEDs to emit light, wherein a second mode of operation is associated with reduced movement and stable physiological condition, wherein the second mode of operation includes a second power management condition including permitting to emit light a first set of the plurality of LEDS that are configured to emit light within a first wavelength range and disallowing from emitting light a second set of the plurality of LEDs that are configured to emit light in a second wavelength range, wherein the first set is different from the second set; and adjusting operation of the plurality of LEDs based on the selected mode.

In some aspects, the techniques described herein relate to a method, wherein the first power management condition includes permitting all LEDs of the plurality of LEDs to emit light.

In some aspects, the techniques described herein relate to a method, wherein the first power management condition includes permitting to emit light a first set of the plurality of LEDs that are configured to emit light within the first wavelength range and disallowing from emitting light a second set of the plurality of LEDs that are configured to emit light within the second wavelength range, wherein the first set is different from the second set, and wherein disallowing the second set from emitting light is based on hardware considerations or physiological considerations.

In some aspects, the techniques described herein relate to a method, the method further including, when operating in the second mode, maintaining a buffer of physiological data associated with light within the second wavelength range after attenuation by tissue of the wearer.

In some aspects, the techniques described herein relate to a method, wherein the first wavelength range corresponds to visible light.

In some aspects, the techniques described herein relate to a method, wherein the second wavelength range corresponds to visible light or infrared wavelengths.

In some aspects, the techniques described herein relate to a method, wherein the at least one physiological signal is associated with the light after attenuation by tissue corresponding to a wrist or a finger of the wearer.

In some aspects, the techniques described herein relate to a method, wherein the at least one physiological signal is associated with light within the first wavelength range after attenuation by tissue of the wearer.

In some aspects, the techniques described herein relate to a method, further including a third mode of operation that is associated with reduced movement and stable physiological condition, wherein the third mode of operation includes a third power management condition including disallowing from emitting light the plurality of LEDs.

In some aspects, the techniques described herein relate to a method, wherein adjusting operation of the plurality of LEDs includes increasing a period of the first mode or decreasing a period of the first mode, or increasing a period of the second mode or decreasing a period of the second mode.

In some aspects, the techniques described herein relate to a method, wherein the first power management condition includes turning on additional motion sensors configured to detect motion of the wearable system.

In some aspects, the techniques described herein relate to a method, wherein the third power management condition includes turning off the at least one motion sensor.

In some aspects, the techniques described herein relate to a method, wherein determining whether the wearer has a stable physiological condition or an unstable physiological condition includes determining whether a physiological threshold condition is satisfied.

In some aspects, the techniques described herein relate to a method, wherein the physiological threshold condition is based in part on a history of physiological data received from one or more detectors.

In some aspects, the techniques described herein relate to a method, wherein determining whether the physiological threshold condition is satisfied includes determining of whether a value, a change, a rate of change, or a change in the rate of change of the physiological data satisfies the physiological threshold condition.

In some aspects, the techniques described herein relate to a method, wherein determining whether the movement condition is elevated or reduced includes determining whether a movement threshold condition is satisfied.

In some aspects, the techniques described herein relate to a method, wherein the movement threshold condition is based in part on a history of movement data received from the at least one motion sensor.

In some aspects, the techniques described herein relate to a method, wherein determining whether the movement threshold condition is satisfied includes determining whether a value, a change, a rate of change, or a change in the rate of change of the movement data satisfies the movement threshold condition.

In some aspects, the techniques described herein relate to a method, wherein the at least one motion sensor is an accelerometer or gyroscope.

In some aspects, the techniques described herein relate to a system configured to implement a method 37-55.

### BRIEF DESCRIPTION OF THE DRAWINGS

FIG. 1A illustrates a first view of an example wearable device including a physiological parameter measurement sensor or module worn on a wrist using straps.

FIG. 1B illustrates a second view of the example wearable device of FIG. 1A worn on the wrist.

FIG. 1C illustrates an example fingertip sensor that can be coupled to the wearable device of the present disclosure.

FIG. 1D illustrates a front view of an example aspect of a physiological parameter measurement sensor or module.

FIG. 1E illustrates an exploded view of an example aspect of a physiological parameter measurement sensor or module.

FIGS. 1F and 1G illustrate light diffusing material fill channels and air venting channels in an opaque frame of an example physiological parameter measurement sensor or module.

FIG. 1H illustrates an example physiological parameter measurement sensor or module and example light paths between emitters and detectors of the module.

FIG. 1I illustrates a perspective view of PCB substrate of a physiological parameter measurement sensor or module with example plethysmograph sensor arrangement.

FIG. 1J illustrates a longitudinal cross-sectional view of an example physiological parameter measurement sensor or module.

FIG. 1K illustrates a longitudinal cross-sectional, that is orthogonal to the view of FIG. 1J, of an example physiological parameter measurement sensor or module.

FIG. 1L illustrates a perspective view of PCB substrate of a physiological parameter measurement sensor or module with example plethysmograph sensor arrangement.

FIG. 1M illustrates a perspective cross-sectional view of an example physiological parameter measurement sensor or module.

FIGS. 1N-10 illustrate an example physiological parameter measurement sensor or module and example light paths between emitters and detectors of the module.

FIGS. 1P-1S illustrate an example physiological parameter measurement sensor or module and example light barriers or light blocks between emitter and detector chambers of the module.

FIG. 1T illustrates an example physiological parameter measurement sensor or module and example light diffusing material and light transmissive lens(es) or cover(s).

FIG. 1U illustrates examples of various monitoring devices.

FIG. 2 illustrates a diagram including a network of non-limiting examples of devices that can communicate with the wearable device disclosed herein.

FIGS. 3A-3B illustrate an example hardware processor and controller for continuous and intermittent monitoring of physiological parameters.

FIG. 4 illustrates an example block diagram corresponding to the systems in FIGS. 3A-3B.

FIG. 5 illustrates an example routine for continuous monitoring of physiological parameters.

FIG. 6 illustrates an example routine for intermittent monitoring of physiological parameters and updating a sampling rate.

### DETAILED DESCRIPTION OF THE PREFERRED EMBODIMENT

Although certain aspects and examples are described below, those of skill in the art will appreciate that the disclosure extends beyond the specifically disclosed aspects and/or uses and obvious modifications and equivalents thereof based on the disclosure herein. Thus, it is intended that the scope of the disclosure herein disclosed should not be limited by any particular aspects described below.

### A. Overview of Wearable Device Including a Physiological Parameter Measurement Sensor or Module

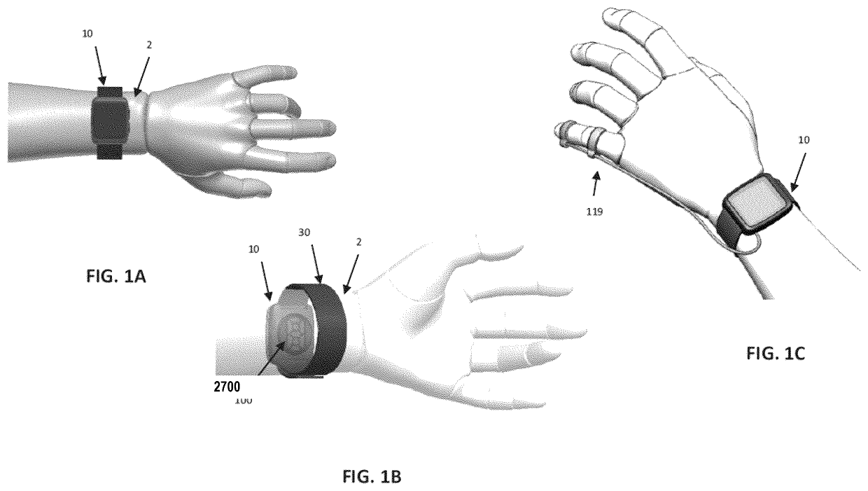

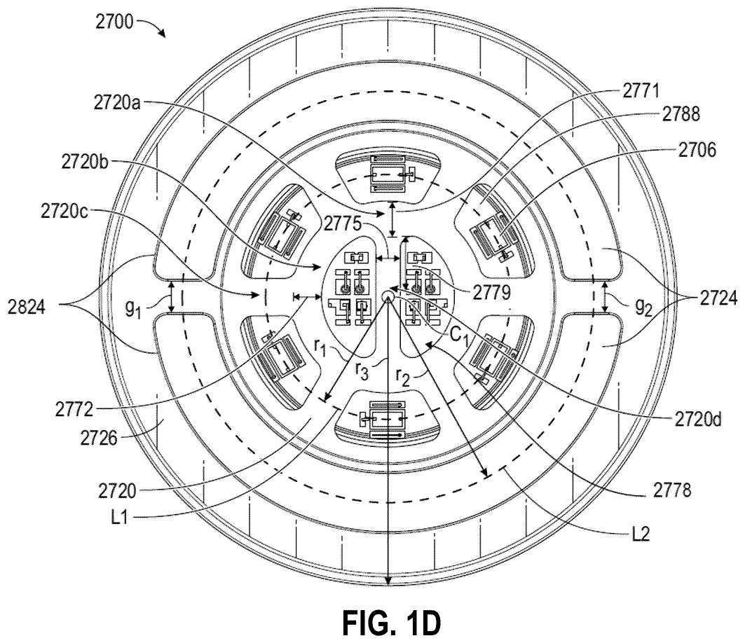

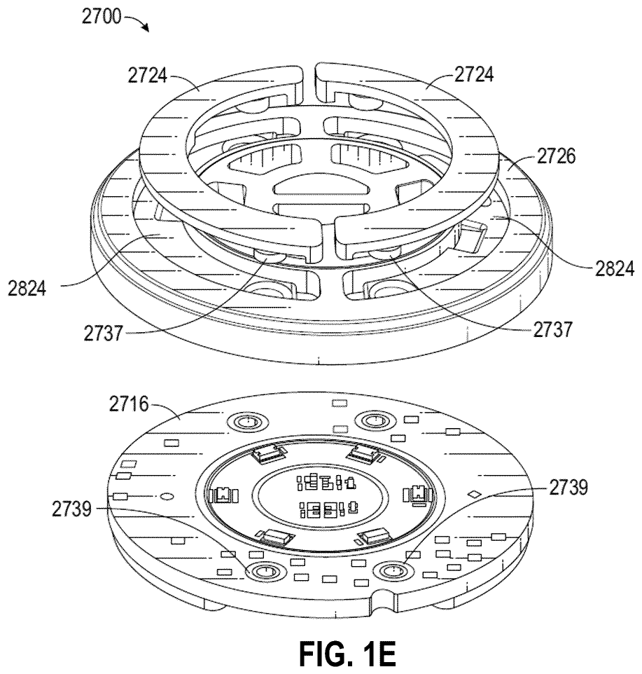

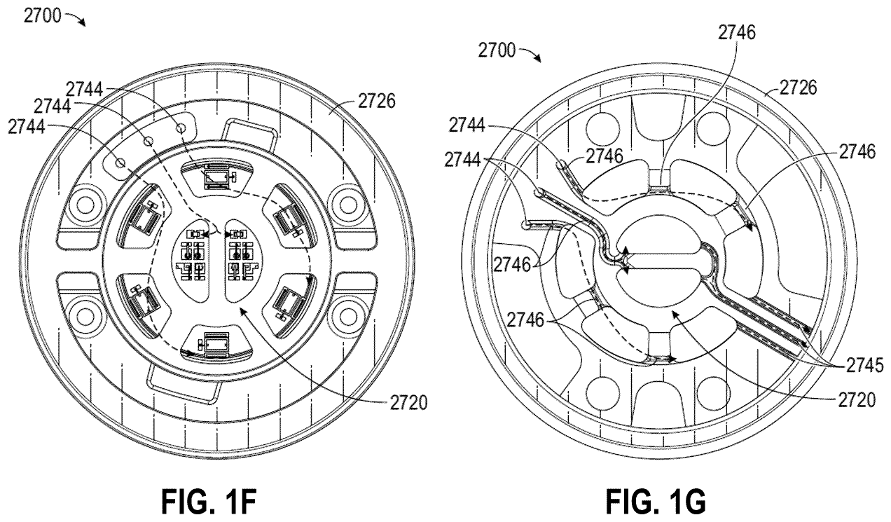

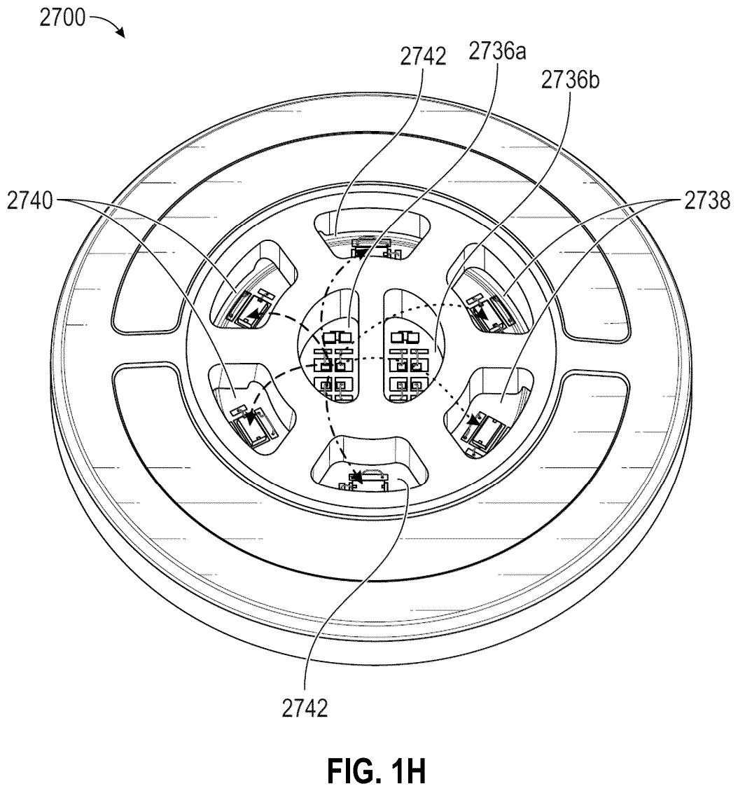

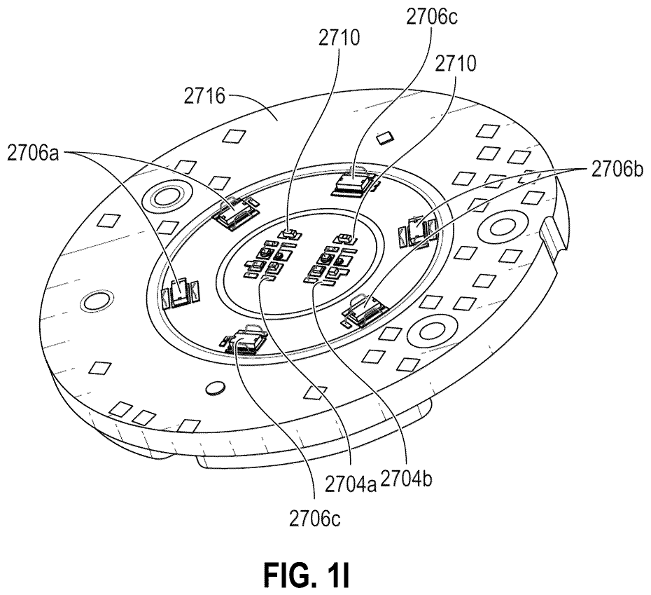

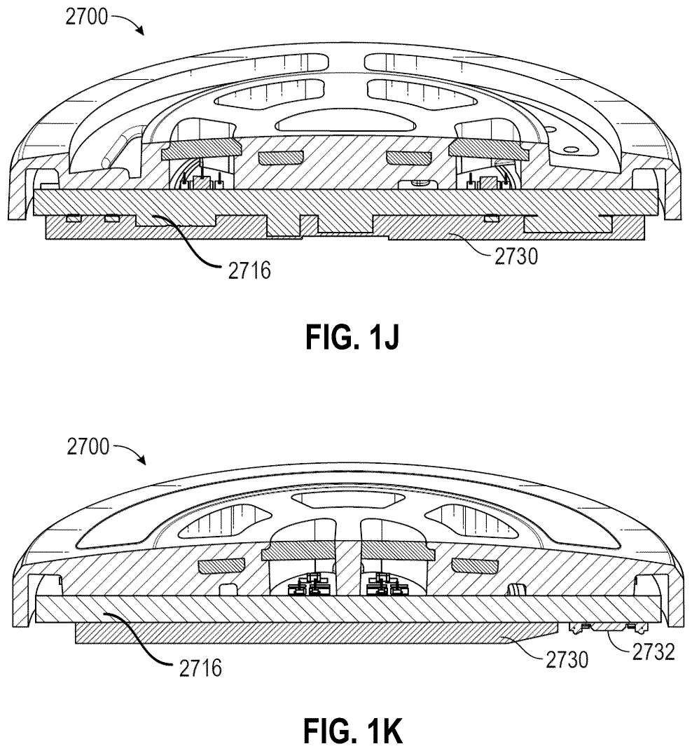

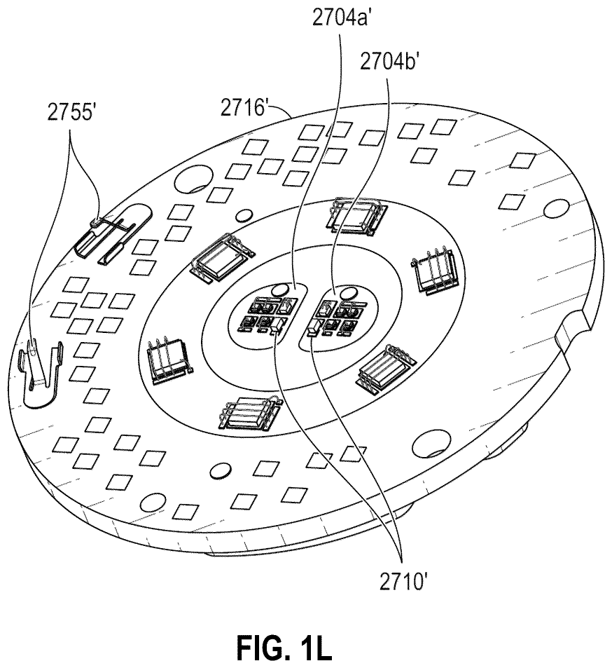

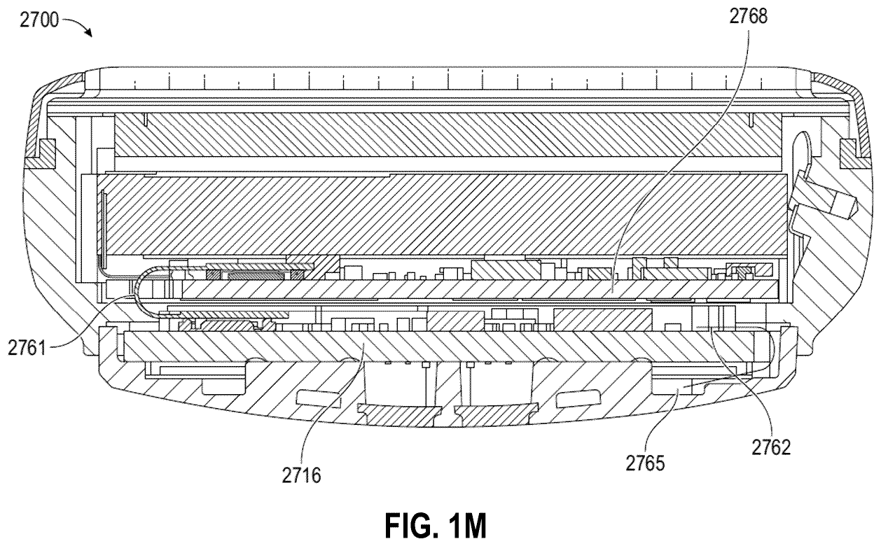

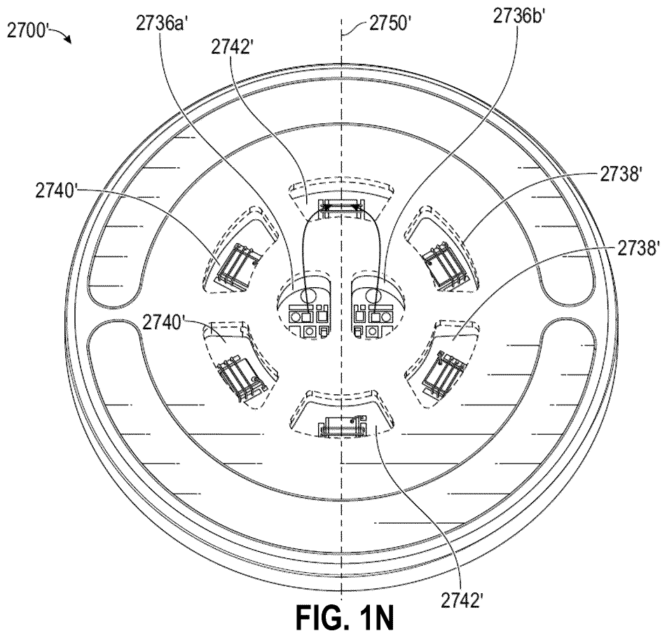

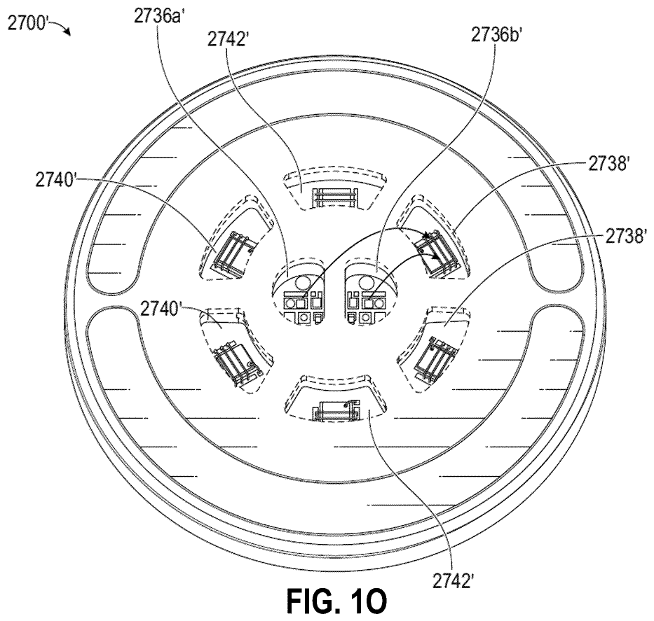

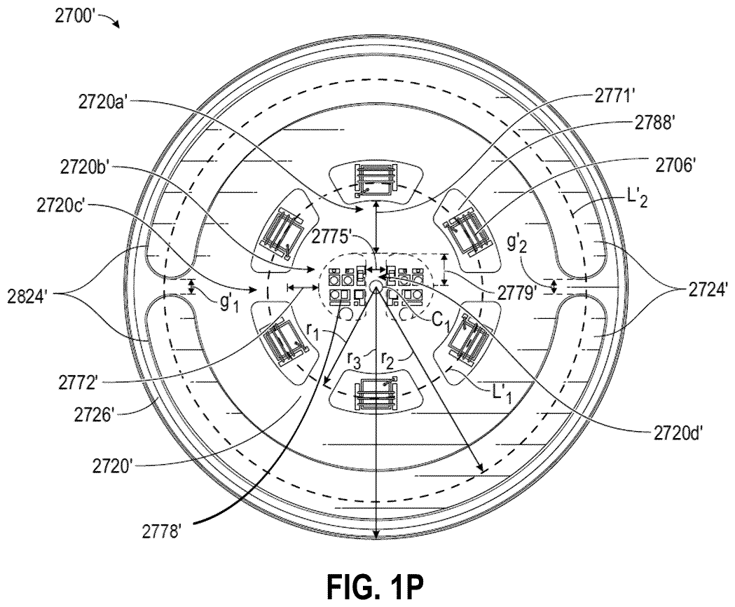

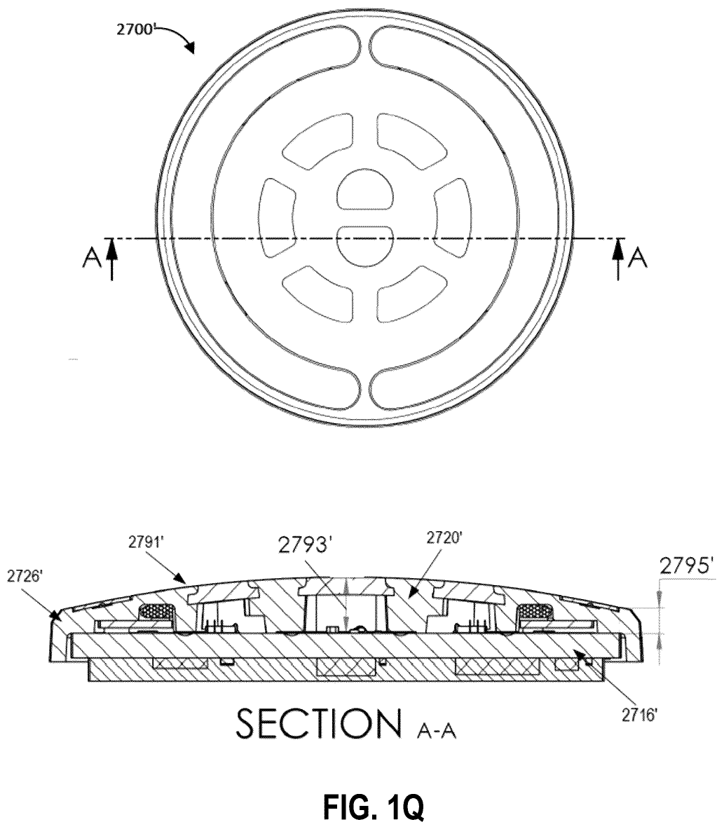

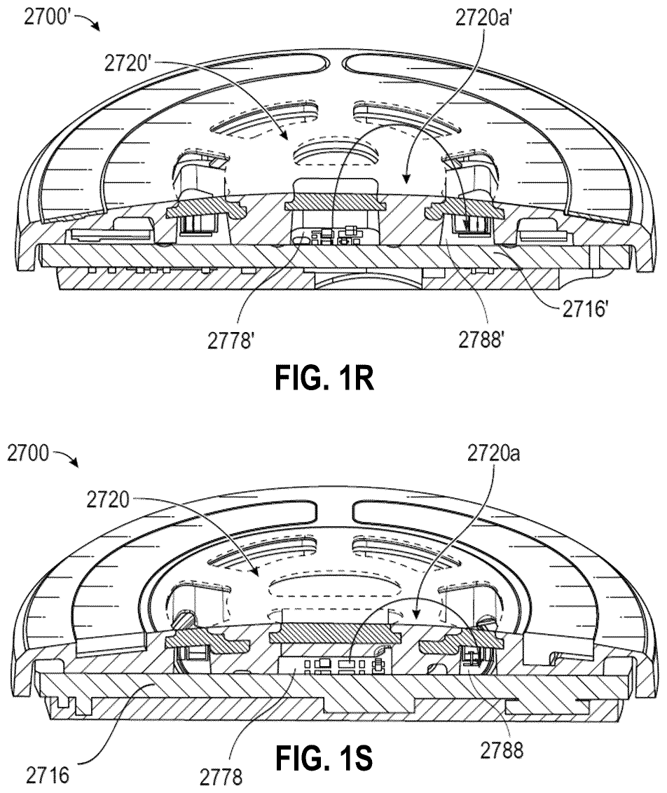

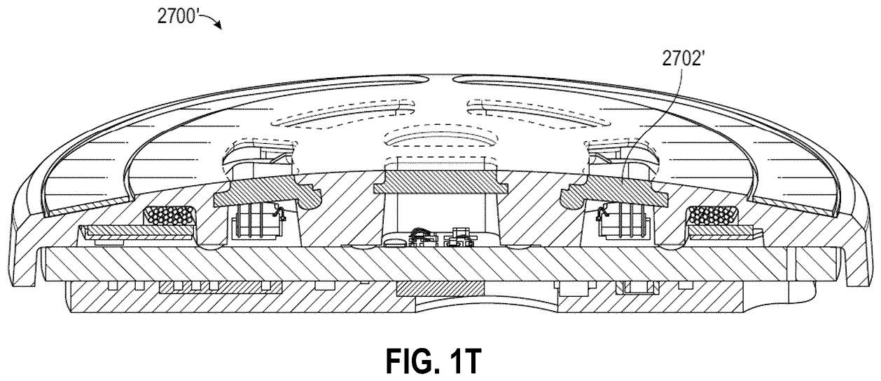

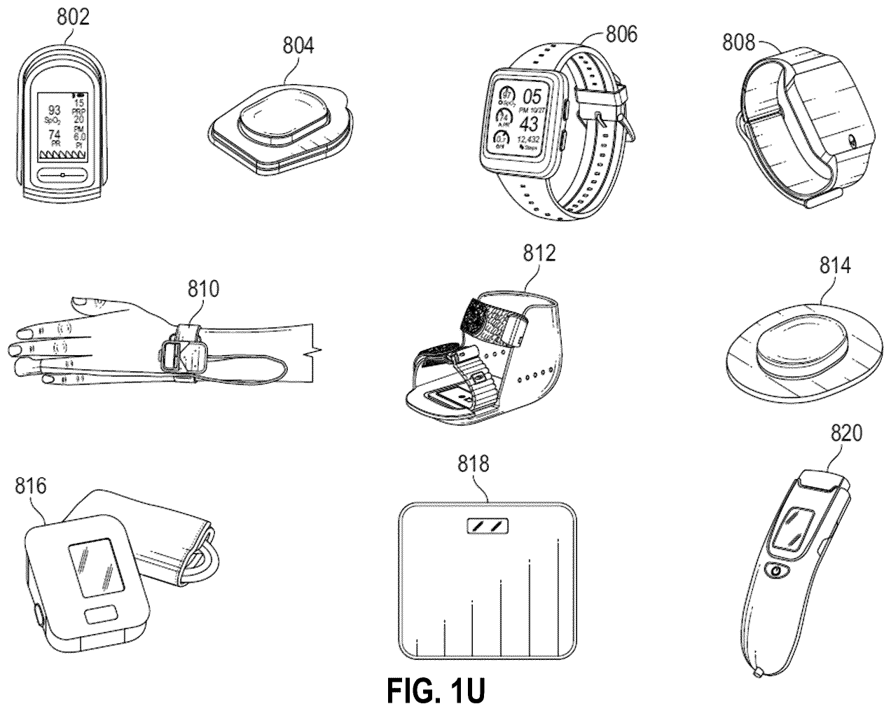

Daily use of a wearable healthcare monitoring device, which can include oximetry- or plethmosmograph-based and/or electrocardiogram (ECG) physiological parameters, can be beneficial to the user. The device, such as a device 10 as shown in FIGS. 1A-1C, can be a wristwatch incorporating a physiological parameter measurement sensor 2700 or a wrist-worn physiological parameter measurement sensor with built-in watch or time-indicating functions. In some cases, the device 10 may be a ring incorporating a physiological parameter measurement sensor 2700 or finger-worn physiological parameter measurement sensor. The device 10 can include an adjustable strap. Accordingly, the user needs not wear an additional sensor when going about daily activities and the appearance of the device attracts less attention from the general public so that the user may feel less self-conscious about wearing a pulse oximeter sensor on the user's body. The user can also connect additional sensors (for example, a fingertip plethysmograph sensor shown in FIG. 1C) and/or other physiological monitoring devices to the wearable device to expand the functionality of the wearable device.

The user can be informed of physiological parameters, such as vital signs including but not limited to pulse rate (sometimes known as heart rate depending on being measured from various sources, such as PPG and ECG), and oxygen saturation by the wearable device 10. The device 10 can display one or more of the measured physiological parameters on its display. The information can be helpful in providing feedback to the user and/or a third party user, for example, a healthcare professional or the user's family member, when the user is exercising, or otherwise for warning the user of possible health-related conditions, including but not limited to changes in the user's physiological parameters in response to medication that is being administered to the user.

As shown in FIGS. 1A-1U, the wearable device 10 can be a watch, which can include a physiological parameter measurement sensor or module 2700 configured to measure an indication of the user's physiological parameters, which can include, for example, PR, respiration rate, SpO2, PVI, PI, Respiration from the pleth (RRp), hydration, glucose, blood pressure, resting PR, desaturation, acoustic data (e.g., microphone and/or phonocardiography), stress, and/or other parameters. The physiological parameter measurement sensor or module 2700 can be an optical sensor. Additionally, the sensor or module 2700 can optionally calculate a wellness index based on more than one individual physiological parameter measured by the module and/or received by the sensor or module 2700 based on externally connected sensors and/or patient monitoring devices. The sensor or module 2700 can perform intermittent and/or continuous monitoring of the measured parameters. The sensor or module 2700 can additionally and/or alternatively perform a spot check of the measured parameters, for example, upon request by the user.

FIGS. 1D-1M illustrate other non-limiting examples of a physiological parameter measurement module 2700 of a wearable device (e.g., wearable device 10 in FIGS. 1A-1C). The module 2700 can incorporate any of the features of the module examples described herein. FIG. 1I illustrates an example arrangement of an optical sensor, including emitters, detectors, and thermistors, on a sensor or module processor substrate 2716. The substrate 2716 can include a printed circuit board (PCB). As shown in FIG. 1I, the substrate 2716 can include a first group of emitters 2704a and a second group of emitters 2704b. Each of the first and second groups of emitters 2704a, 2704b can include four emitters (or optionally a different number of emitters, such as three, six or eight emitters or any other number of emitters as required or desired). Each of the emitters of the first and second groups of emitters 2704a, 2704b may comprise an LED.

The emitters in the first and second group of emitters 2704a, 2704b can include operational and/or structural features. For example, the first and second group of emitters 2704a, 2704b can be configured to emit a plurality of (for example, three, four, or more) wavelengths. In some aspects, each of the emitters (e.g., within a group) can be configured to emit light of a different wavelength than the other emitters (e.g., of that group). Alternatively, one or more of the emitters can emit light of more than one wavelength. The emitters in the first and second group of emitters 2704a, 2704b can emit at least the first, second, third, and fourth wavelengths.

Each of the emitters in the first group of emitters 2704a may be located within close proximity to each of the other emitters in the first group of emitters 2704a. For example, each of the emitters of the first group of emitters 2704a may be located on the PCB between 0.2 mm and 2 mm from each of the other emitters in the first group of emitters 2704a. For example, each of the emitters of the first group of emitters 2704a may be located on the PCB about 0.5 mm from each of the other emitters of the first group of emitters 2704a. In some aspects, each of the emitters in the first group of emitters 2704a are positioned such that no more than a certain distance is between each of the emitters in the first group of emitters 2704a, such as 0.5 mm, 1 mm, 1.5 mm and/or 2 mm, or any other distance as required or desired. Each of the emitters in the second group of emitters 2704b may be located within close proximity to each of the other emitters in the second group of emitters 2704b, for example as described above with reference to the first group of emitters 2704a.

Each of the two groups of the emitters 2704a, 2704b can be surrounded by a first light barrier and form emitter chambers for the group of emitters 2704a and group of emitters 2704b, respectively. The first and second groups of emitters 2704a, 2704b can be located a distance from each other on a first side of a substrate 2716. The substrate 2716 can include one or more temperature sensor(s) (such as a thermistor) 2710 as described above located on the first side of the substrate 2716. One temperature sensor 2710 can be near the first group of emitters 2704a within the respective emitter chamber. Another temperature sensor 2710 can be near the second group of emitters 2704b within the respective emitter chamber.

The substrate 2716 can be circular in shape, although the shape of the PCB is not limiting. The two groups of the emitters 2704a, 2704b can be located on different parts of the first side of the substrate 2716 divided along a center line of the circle. Each of the two groups of the emitters 2704a, 2704b can be surrounded by a first light barrier and form an emitter chamber.

The first and second groups of emitters 2704a, 2704b can be surrounded by detectors 2706. As described in greater detail with reference to FIG. 1D, the detectors 2706 can be positioned on the substrate 2716 in a substantially circular or annular arrangement. The detectors 2706 may surround and/or enclose the first and second groups of 2704a, 2704b. Each of the detectors may be a similar or same distance from a geometric center of the substrate 2716 and/or the sensor or module 2700. In some aspects, the detectors 2706 may be rectangular including longer sides and shorter sides. The detectors 2706 may be positioned on the substrate 2716 such that a long side of each detector is orthogonal to a radius of the substrate 2716. In the illustrated example, the substrate 2716 includes six detectors coupled to the substrate 2716, but the total number of detectors can vary.

The detectors 2706b can be the far detectors for the first group of emitters 2704a. The detectors 2706a can be the near detectors for the first group of emitters 2704a. The detectors 2706a can be the far detectors for the second group of emitters 2704b. The detectors 2706b can be the near detectors for the second group of emitters 2704b. The detectors 2706c can be the intermediate detectors for the first and second groups of emitters 2704a, 2704b. Accordingly, each detector 2706a, 2706b, 2706c can receive two signals for each wavelength emitted by the first and second groups of emitters 2704a, 2704b respectively. As described above, signals outputted by the far, near and intermediate detectors can provide different information due to the different light paths, which can travel through different areas of the tissue.

In some aspects, the sensor or module processor may evaluate the various signals outputted by the detectors for example by comparing the signal quality of the detectors. The sensor or module processor may select less than all of the detector signals for processing for each of the far, near and intermediate detectors. For example, the sensor or module processor may rely on signals from one or two detectors from the four possible far detectors, and one or two detectors from the four possible near detectors, and one or two detectors from the four possible intermediate detectors.

In addition, the far detectors for each group of emitters 2704a, 2704b can detect the light emitted by the respective group of emitters 2704a, 2704b, for example, light of the fourth wavelength and another wavelength, and attenuated by tissue to provide an indication of the wearer's hydration status as described herein.

The detectors 2706a, 2706b, 2706c can be separated or partitioned into six detector regions. Each detector region can include one detector, or any other number of detectors. Each detector region can form a detector chamber surrounded by light barriers. As described above, the sensor or module processor can process signals from a particular emitter and received at the detectors within the same detector region as one signal source.

The module 2700 can include individual lenses or covers or a combination of individual emitter chamber covering lenses or covers and a lens or cover covering a plurality of detector chambers. The lenses or covers may be polycarbonate. The tissue-facing surface of the module 2700 can include a continuous convex curvature.

FIG. 1L illustrates another example arrangement of an optical sensor, including emitters, detectors, and thermistors, on a sensor or module processor substrate 2716′. The substrate 2716′ of FIG. 1L can include structural and/or operational features similar to those discussed above with reference to FIG. 1I. As shown in FIG. 1L, each of the first and second groups of emitters 2704a′, 2704b′ can include five emitters (or optionally a different number of emitters as required or desired). Each of the emitters of the first and second groups of emitters 2704a′, 2704b′ may comprise an LED and can be configured to emit light at various wavelengths such as any of the wavelengths discussed herein, for example, a first wavelength of about 525 nm to about 650 nm (such as about 525 nm or about 580 nm or about 645 nm), a second wavelength from about 620 nm to about 660 nm (such as about 625 nm), a third wavelength from about 650 nm to about 670 nm (such as about 660 nm), a fourth wavelength from about 900 nm to about 910 nm, and a fifth wavelength at about 970 nm.

As shown in FIG. 1L, the substrate 2716′ can include spring contacts 2755′ for facilitating physical and/or electrical connection between the substrate 2716′ and electrodes (e.g., electrodes 2724 shown in FIG. 1E, for example).

As shown in FIGS. 1J-1K, on the second side of the substrate 2716, which faces away from the cover, the substrate 2716 can be covered by melt plastic or other suitable electronics protective material 2730 (similar to the protective material 130 disclosed herein) except that a flex connector 2732 can remain exposed. The flex connector 2732 can be configured to connect the module 2700 electrically to the wearable device incorporating the module 2700.

FIG. 1M illustrates a perspective cross-sectional view of an example physiological parameter measurement sensor or module 2700. As shown, the sensor or module 2700 can include a first circuit board (e.g., substrate 2716) and a second circuit board (e.g., instrument PCB 2768. The sensor or module 2700 can include an induction coil 2765. The induction coil 2765 can be electrically and/or physically connected to the substrate 2716, for example via solder and/or a flex connector connection 2762. The induction coil can provide a voltage to the substrate 2716, for example via the solder and/or flex connector connection 2762. The substrate 2716 can include charging circuitry which can be configured to receive and/or control a voltage received from the induction coil 2765. The substrate 2716 can be configured to transfer voltage received from the induction coil 2765 to the instrument PCB 2768, for example via a flex connector 2761. The instrument PCB 2768 can be configured to transfer voltage received from the substrate 2716 to a battery or to other components of the sensor or module 2700 as required or desired.

FIGS. 1N-10 illustrate an example physiological parameter measurement sensor or module 2700′ and example light paths between emitters and detectors of the module 2700′.

FIG. 1N illustrates an example arrangement of emitter and detector chambers of the sensor or module 2700′. As shown, the sensor or module 2700′ can include a first emitter chamber 2736a′ enclosing a first emitter group comprising one or more emitters, a second emitter chamber 2736b′ enclosing a second emitter group comprising one or more emitters, one or more first detector chambers 2740′, one or more second detector chambers 2742′, and one or more third detector chambers 2738′. In some aspects, each detector chamber may enclose one detector.

The first emitter group of the first emitter chamber 2736a′ may comprise the same number and type of emitters as the second emitter group of the second emitter chamber 2736b′. In other words, each emitter of the first emitter group may correspond to an emitter of the same type (e.g., same wavelength) of the second emitter group. The emitters of the first emitter group may be arranged in a configuration that mirrors the emitters of the second emitter group across a centerline 2750 of the sensor or module 2700′ as shown in FIG. 1N. For example, each emitter of the first group of emitters may be located a distance away from a centerline 2750 of the sensor or module 2700′ that is a same distance that a corresponding emitter of the second group of emitters is located away from the centerline 2750 of the sensor or module 2700′. For example, the first and second emitter groups may each include an emitter that emits light of a first wavelength and that are positioned at locations that are mirror images of each other across a centerline 2750 of the sensor or module 2700′. Additionally, the first and second emitter groups may each include an emitter that emits light of a second wavelength and that are positioned at locations that are mirror images of each other across a centerline 2750 of the sensor or module 2700′. Each of the emitters of the first emitter group may correspond to an emitter of the second emitter group located at a mirror image position, and vice versa.

The one or more second detector chambers 2742′ may be bisected by a centerline 2750 of the sensor or module 2700′. Each of the detectors of the respective one or more second detector chambers 2742′ may be bisected by a centerline 2750 of the sensor or module 2700′. In other words, the one or more second detector chambers 2742′ and the respective detectors and the sensor or module 2700′ may each share a same (e.g., parallel) centerline 2750. The sensor or module 2700′ may be oriented (e.g., rotated) with respect to the tissue of a wearer in any orientation. In an example implementation where the sensor or module 2700′ is worn on a wrist of a user, the sensor or module 2700′ may be rotated in any direction with respect to the wrist or forearm of the wearer. In one example configuration, the sensor or module 2700′ may be oriented with respect to the forearm (or other body part) of a wearer such that the centerline 2750′ of the sensor or module is perpendicular to a line extending along a length of the forearm of the wearer (e.g., from the elbow to the wrist). Advantageously, such a configuration may improve physiological measurements by facilitating light emitted from the emitter chambers and detected at the detector chambers (e.g., light travelling from emitter chamber 2736a′ to detector chamber 2738′) to penetrate into soft tissue of the wearer (e.g., blood vessels) rather than other tissues such as bone. In another example configuration, the sensor or module 2700′ may be oriented with respect to the forearm (or other body part) of a wearer such that the centerline 2750′ of the sensor or module is parallel to a line extending along a length of the forearm of the wearer (e.g., from the elbow to the wrist). Advantageously, such a configuration may improve physiological measurements by facilitating light emitted from the emitter chambers and detected at the detector chambers (e.g., light travelling from emitter chamber 2736a′ to detector chamber 2742′) to penetrate into soft tissue of the wearer (e.g., blood vessels) rather than other tissues such as bone.

As shown in FIG. 1N, emitters of the first and second emitter groups that correspond to each other (e.g., emit the same wavelength and mirror each other) may each emit light that travels along respective paths to the detectors of the one or more second detector chambers 2742′. The respective paths of light from the corresponding emitters may be of equal length. This may be because the corresponding emitters are each positioned an equal distance away from a detector of a chamber 2742′. The corresponding emitters may each be an equal distance away from a detector of a chamber 2742′ because they are positioned at mirror images of each other across a centerline 2750 of the sensor or module 2700′ that bisects the one or more second detector chambers 2472′ and respective detectors.

The one or more second detector chambers 2742′ and their respective detectors may be used, at least in part, for calibration, for example to characterize the emitters, by providing known information such as a known ratio. For example, information corresponding to a wavelength detected at a detector of a chamber 2742′ from an emitter of the first group of emitters may be similar or the same as information corresponding to that wavelength detected at the detector of the chamber 2742′ from an emitter of the second group of emitters and a comparison (e.g., subtracting, dividing, etc.) of the information resulting from the first and second groups of emitters may yield a known number such as zero or one because the corresponding emitters from the first and second emitter groups may be an equal distance from the detector of chamber 2742′ and light emitted therefrom may travel a same distance to the detector of chamber 2742′. As an example of normalization, ratios of wavelengths detected at detectors of chambers 2738′, 2740′ may be normalized (e.g., divided by) ratios of wavelengths detected at detectors of chambers 2742′. In instances where the information resulting from detection of light from the first and second groups of emitters is not the same or is substantially different (e.g., as a result of emission intensity variations or other such discrepancies) the information may be adjusted or normalized (e.g., calibrated) to account for such differences. This normalization or on-board calibration or characterization of the emitters may improve accuracy of the physiological measurements and provide for continuous calibration or normalization during measurements. In some aspects, a processor may be configured to calibrate or normalize the physiological parameter measurement of the sensor continuously. In some aspects, a processor may be configured to calibrate or normalize the physiological parameter measurement of the sensor while the optical physiological sensor measures physiological parameters of the wearer.

FIG. 1O illustrates an example arrangement of emitter and detector chambers of the sensor or module 2700′. As shown, the sensor or module 2700′ can include a first emitter chamber 2736a′, a second emitter chamber 2736b′, one or more first detector chambers 2740′, one or more second detector chambers 2742′, and one or more third detector chambers 2738′, for example as discussed elsewhere herein.

The first and second emitter chambers 2736a′, 2736b′ may be located at non-equal distances away from each of the chambers of the one or more detector chambers 2738′, 2740′. Thus, with respect to each detector chamber of the chambers 2738′, 2740′, the first and second emitter chamber 2736a′, 2736b′, may each be a “near” or “far” emitter chamber. In other words, each detector of the detector chambers 2738′, 2740′ may detect light, of any given wavelength, from both a “near” emitter and a “far” emitter, with the near and far emitters being included in either the first or second emitter group, respectively.

As an example, as shown in FIG. 1O, light of a given wavelength may travel along a path from an emitter in the first emitter group to the detector of detector chamber 2738′ and light of the same wavelength may travel along a path from an emitter in the second emitter group to the same detector. The light from the first emitter group may travel along a longer path than light from the second emitter group before reaching the detector of chamber 2738′. Thus, for any detector of detector chambers 2738′ or 2740′, the detector may receive light of a given wavelength from both a near (e.g., proximal) emitter and a far (e.g., distal) emitter. This may not be the case for detectors of chambers 2742′ because the first and second emitter groups may each be located a same distance away from any given detector of detector chambers 2742′, as described herein.

For convenience, the terms “proximal” and “distal” may be used herein to describe structures relative to any of the detector chambers or their respective detectors. For example, an emitter may be proximal to a detector chamber of the first detector chambers and distal to a detector of the second detector chambers. The term “distal” refers to one or more emitters that are farther away from a detector chamber than at least some of the other emitters. The term “proximal” refers to one or more emitters that are closer to a detector chamber than at least some of the other emitters. The term “proximal emitter” may be used interchangeably with “near emitter” and the term “distal emitter” may be used interchangeably with “far emitter”.

A single emitter may be both proximal to one detector and distal to another detector. For example, an emitter may be a proximal emitter relative to a detector of the first detector chambers and may be a distal emitter relative to a detector of the second detector chambers.

Light of a given wavelength that is detected at a detector may provide different information depending on the length of the path it has travelled from the emitter (e.g., along a long path from a distal emitter or along a short path from a proximal emitter). For example, light that has travelled along a long path from a distal emitter may penetrate deeper into the tissue of a wearer of the device and may provide information pertaining to pulsatile blood flow or constituents. The use of a proximal and distal emitter for each wavelength may improve accuracy of the measurement, for example information pertaining to light that has travelled along a long path from a distal emitter may be normalized by (e.g., divided by) information pertaining to light that has travelled along a short path from a proximal emitter.

FIGS. 1P-1S illustrate an example physiological parameter measurement sensor or module 2700′ and example light barriers or light blocks between emitter and detector chambers of the module 2700′.

FIG. 1P is a front view of an example aspect of a sensor or module 2700′. The sensor or module 2700′ includes an opaque frame opaque frame 2726′, one or more electrodes 2724′, one or more detector chambers 2788′, one or more emitter chambers 2778′, and a light barrier construct 2720′.

The opaque frame 2726′ can include one or more materials configured to prevent or block the transmission of light. In some aspects, the opaque frame 2726′ may form a single integrated unit. In some aspects, the opaque frame 2726′ may be formed of a continuous material. The light barrier construct 2720′ can include one or more materials configured to prevent or block the transmission of light. In some aspects, the light barrier construct 2720′ may form a single integrated unit. In some aspects, the light barrier construct 2720′ may be formed of a continuous material. In some aspects, the light barrier construct 2720′ and the opaque frame 2726′ may form a single integrated unit. In some aspects, the light barrier construct 2720′ and the opaque frame 2726′ may be separably connected.

The light barrier construct 2720′ may include one or more light barriers, such as light barriers 2720a′, 2720b′, 2720c′, 2720d′, which are provided as non-limiting examples. In some aspects, light barriers may be also be referred to as light blocks herein. The light barriers may form one or more portions of the light barrier construct 2720′. The light barrier construct 2720′ (or light barrier portions thereof) may prevent light from passing therethrough. The light barrier construct 2720′ may include spaces between various light barriers which may define one or more chambers (e.g., detector chambers 2788′, emitter chambers 2778′). In some aspects, the one or more chambers (e.g., detector chambers 2788′, emitter chambers 2778′) may be enclosed by the light barrier construct 2720′ or light barrier portions thereof, a surface of a substrate (e.g., PCB), and a lens or cover. In some aspects, light may only enter the chambers through the lens or cover.

An example of a light barrier is provided with reference to example light barrier 2720a′. Light barrier 2720a′ forms a portion of light barrier construct 2720′. Light barrier 2720a′ may prevent (e.g., block) light from passing therethrough between adjacent chambers. For example, light barrier 2720a′ may prevent light from passing through the light barrier construct 2720′ between an emitter chamber 2778′ and a detector chamber 2788′. Light barrier 2720a′, or portions thereof, may include a width 2771′. In some aspects, width 2771′ may be less than about 3.30 mm. In some aspects, width 2771′ may be less than about 3.25 mm. In some aspects, width 2771′ may be less than about 3.20 mm. In some aspects, width 2771′ may be about 3.24 mm. In some aspects, the width 2771′ may be greater (e.g., larger) than length 2779. In some aspects, width 2771′ may be less than about 165% of length 2779. In some aspects, width 2771′ may be less than about 160% of length 2779. In some aspects, width 2771′ may be less than about 155% of length 2779. In some aspects, width 2771′ may be about 160% of length 2779. Advantageously, a greater width 2771′ (e.g., a wider light barrier separating the emitter chambers 2778′ and detector chambers 2788′) may cause light emitted from the emitter chambers 2778′ to travel a greater distance before reaching the detector chambers 2788′. Light that travels a greater distance may penetrate deeper into the tissue of the wearer which may improve accuracy of a physiological measurement.

Another example of a light barrier is provided with reference to example light barrier 2720b′. Light barrier 2720b′ forms a portion of light barrier construct 2720′. Light barrier 2720b′ may prevent (e.g., block) light from passing therethrough between adjacent chambers. For example, light barrier 2720b′ may prevent light from passing through the light barrier construct 2720′ between an emitter chamber 2778′ and a detector chamber 2788′. Light barrier 2720b′, or portions thereof, may include a width 2772′. In some aspects, width 2772′ may be less than about 1.65 mm. In some aspects, width 2772′ may be less than about 1.60 mm. In some aspects, width 2772′ may be less than about 1.55 mm. In some aspects, width 2772′ may be about 1.59 mm. In some aspects, the width 2772′ may be less (e.g., smaller) than width 2771′. In some aspects, width 2772′ may be less than about 60% of width 2771′. In some aspects, width 2772′ may be less than about 55% of width 2771′. In some aspects, width 2772′ may be less than about 50% of width 2771′. In some aspects, width 2772′ may be about 49% of width 2771′. Advantageously, a greater width 2772′ may cause light emitted from the emitter chambers 2778′ to travel a greater distance before reaching the detector chambers 2788′. Light that travels a greater distance may penetrate deeper into the tissue of the wearer which may improve accuracy of a physiological measurement

Another example of a light barrier is provided with reference to example light barrier 2720c′. Light barrier 2720c′ forms a portion of light barrier construct 2720′. Light barrier 2720c′ may prevent (e.g., block) light from passing therethrough between adjacent chambers. For example, light barrier 2720c′ may prevent light from passing through the light barrier construct 2720′ between adjacent detector chamber 2788′.

Another example of a light barrier is provided with reference to example light barrier 2720d′. Light barrier 2720d′ forms a portion of light barrier construct 2720′. Light barrier 2720d′ may prevent (e.g., block) light from passing therethrough between adjacent chambers. For example, light barrier 2720d′ may prevent light from passing through the light barrier construct 2720′ between adjacent emitter chambers 2778′. In some aspects, light barrier 2720d′ may have a width 2775′ separating adjacent emitter chambers of less than about 1.40 mm. In some aspects, width 2775′ may be less than about 1.35 mm. In some aspects, width 2775′ may be less than about 1.30 mm. In some aspects, width 2775′ may be about 1.28 mm. In some aspects, width 2775′ may be less (e.g., smaller) than width 2771′. In some aspects, width 2775′ may be less than about 50% of width 2771′. In some aspects, width 2775′ may be less than about 45% of width 2771′. In some aspects, width 2775′ may be less than about 40% of width 2771′. In some aspects, width 2775′ may be less than about 35% of width 2771′. In some aspects, width 2775′ may be about 39.5% of width 2771′.

The emitter chambers 2778′ are positioned within a central region of the sensor or module 2700′. The emitter chambers 2778′ may be positioned adjacent to one another across a centerline of the sensor or module 2700′ as described in greater detail with reference to FIG. 1N, for example. The emitter chambers 2778′ may be positioned adjacent the center point C′₁. Each of the emitter chambers 2778′ may be a similar size and/or shape. The emitter chambers 2778′ may be separated, at least in part, by light barrier 2720d′ of the light barrier construct 2720′. In some aspects, as shown in this example, the light barrier 2720d′ may form an entire distance between emitter chambers 2778′. For example, emitter chambers 2778′ may be separated by only the light barrier 2720d′ such that other components (e.g., detectors, detector chambers, etc.) are not positioned between the emitter chambers 2778′.

A portion of the emitter chambers 2778′ may extend a length 2779 away from center point C′₁. In some aspects, length 2779 may be less than about 2.15 mm. In some aspects, length 2779 may be less than about 2.10 mm. In some aspects, length 2779 may be less than about 2.05 mm. In some aspects, length 2779 may be less than about 2.0 mm. In some aspects, length 2779 may be about 2.02 mm. In some aspects, the length 2779 may be less (e.g., smaller) than a width of a light barrier separating an emitter chamber from a detector chamber such as width 2771′. In some aspects, length 2779 may be less than about 70% of width 2771′. In some aspects, length 2779 may be less than about 65% of width 2771′. In some aspects, length 2779 may be less than about 60% of width 2771′. In some aspects, length 2779 may be about 62.3% of width 2771′.

As shown in this example aspect, the detector chambers 2788′ are arranged in a substantially circular pattern. Each of the detector chambers 2788′ houses a detector 2706 positioned on a substrate (e.g., PCB) in a substantially circular or annular pattern. The detectors 2706 may be positioned in a central region of each of the respective detector chambers 2778′. The detector chambers 2788′ are arranged along a ring defined by ring L′₁. In some aspects, such as shown in this example aspect, detectors 2706 of respective detector chambers 2788′ may also be arranged along a same ring along which the detector chambers 2788′ are arranged (such as in aspects where detectors are positioned in a central region of respective chambers). The ring L′₁ may intersect a central region of the detector chambers 2788′. In this example aspect, the ring L′₁ encloses an entirety of the emitter chambers emitter chambers 2778′ such that the emitter chambers 2778′ are positioned within an interior region (e.g., a central region) of the ring L′₁ defined by the detector chambers 2788′. In some aspects, each of the detector chambers 2788′ (and corresponding detectors 2706 within respective detector chambers 2788′) may be positioned at a substantially similar or same distance away from the center point C′₁ (e.g., center of sensor or module 2700′). In some aspects, the detectors 2706 may be rectangular including longer sides and shorter sides. The detectors 2706 may be positioned on a substrate of the sensor or module 2700′ such that a long side of each detector is orthogonal to a radius extending away from center point C′₁ (e.g., radius r′₁, radius r′₂, radius r′₃). Advantageously, orienting the detectors 2706 on the sensor or module 2700′ in an annular arrangement with a long side of the detectors 2706 orthogonal the center point C′₁ may improve an accuracy of physiological measurements by ensuring that light from emitters travels along a known path length from emitters to the detectors 2706 and may also reduce processing requirements of the sensor or module 2700′ by reducing the amount of variables (e.g., number of light path lengths) required to process in order to determine physiological data.

The electrodes 2724′ can include a reference electrode and a negative electrode (and/or a positive electrode). In some aspects, a wearable device such as a watch incorporating the sensor or module 2700′ can include another ECG electrode (e.g., a positive electrode) located on the housing of the wearable device configured to make contact with the wearer's skin. In some configurations, a surface of the electrodes 2724′ may be flush with a surface of the opaque frame 2726′.

The electrodes 2724′ are positioned within or along a portion of the opaque frame 2726′ such as shown in FIG. 1E for example. In some aspects, the electrodes 2724′ can be substantially semicircular. In some aspects, the electrodes 2724′ can be substantially semi-annular. In the example aspect shown, each of the electrodes 2724′ forms a substantial half annulus. Advantageously, an annular shaped electrode may improve contact with the skin of a wearer (e.g., by contacting a diverse area of skin) while simultaneously reducing the amount of surface area of the electrode. In some aspects, each of the electrodes 2724′ may be a similar size and/or shape. In some aspects, the electrodes 2724′ may be various sizes and/or shapes. In this example aspect, the electrodes 2724′ are positioned within the sensor or module 2700′ (e.g., within the opaque frame 2726′) along ring defined by L′₂. In various aspects described herein, the ring L′₂ may include various radii which may advantageously provide improved contact between the electrodes 2724′ and the skin of a wearer of the device. In some implementations, frame 2726′ includes recesses that 2824′ are sized and/or shaped to accommodate the ECG electrodes 2724′. In some implementations, recesses 2824′ have a depth (for example, measured from a plane of the frame 2726′) that is substantially equal to a thickness of the ECG electrodes 2724′. In some implementations, recesses 2824′ have a size and/or shape that matches a size and/or shape of the ECG electrodes 2724′. For example, in some implementations in which the ECG electrodes have a semi-annular shape, the recesses 2824′ can have a semi-annular shape.

The opaque frame 2726′ includes one or more gaps (e.g., g′₁, g′₂) between electrodes 2724′. The gaps g′₁, g′₂, (or other portions of the opaque frame 2726′) may electrically insulate each of the electrodes 2724′ from one another. Each of the electrodes 2724′ includes a curved edge along a portion of respective gaps g′₁, g′₂. In some aspects, the gaps g′₁, g′₂, may be a similar or a same size. In some aspects, the gaps g′₁, g′₂, may be a different size than each other. In some aspects, the gaps g′₁, g′₂, may be less than about 0.6 mm. In some aspects, the gaps g′₁, g′₂, may be less than about 0.65 mm. In some aspects, the gaps g′₁, g′₂, may be less than about 0.7 mm. In some aspects, the gaps g′₁, g′₂, may be about 0.62 mm. As discussed above, in some implementations the frame 2726′ includes recesses 2824′ sized and/or shaped to receive the ECG electrodes 2724′. In some implementations, each of such recesses 2824′ includes first and second ends, the first ends of the recesses 2824′ are separated from one another by gap g′₁, and the second ends of the recesses 2824′ are separated from one another by gap g′₂ (see FIG. 1P). In some implementations, such as that illustrated in at least FIG. 1P, ends of the recesses 2824′ and/or ends of ECG electrodes 2724′ have a rounded shape.

The ring L′₁ may be concentric with an outer perimeter of the sensor or module 2700′. The ring L′₂ may be concentric with an outer perimeter of the sensor or module 2700′. The ring L′₂ may be concentric with a ring defined by positions of the detector chambers 2788′ such as ring L′₁. Center point C′₁ may define a geometric center of ring L′₁. Center point C′₁ may define a geometric center of ring L′₂. Center point C′₁ may define a geometric center of an outer perimeter of the sensor or module 2700′. In some aspects, such as shown in FIG. 1P, each of L′₁, L′₂, and an outer perimeter of the sensor or module 2700′ are concentric with each other and share a same geometric center shown as C′₁.

The ring L′₁ may include a radius r′₁. In some aspects, radius r′₁ may be less than about 6.5 mm. In some aspects, radius r′₁ may be less than about 6.45 mm. In some aspects, radius r′₁ may be less than about 6.40 mm. In some aspects, radius r′₁ may be about 6.40 mm. In some aspects, the radius r′₁ may be less (e.g., smaller) than radius r′₂. In some aspects, radius r′₁ may be less than about 60% of r′₂. In some aspects, radius r′₁ may be less than about 55% of r′₂. In some aspects, radius r′₁ may be less than about 50% of r′₂. In some aspects, radius r′₁ may be about 50.9% of r′₂. In some aspects, the radius r′₁ may be less (e.g., smaller) than radius r′₃. In some aspects, radius r′₁ may be less than about 40% of r′₃. In some aspects, radius r′₁ may be less than about 45% of r′₃. In some aspects, radius r′₁ may be less than about 50% of r′₃. In some aspects, radius r′₁ may be about 42% of r′₃.

The ring L′₂ may include a radius r′₂. In some aspects, radius r′₂ may be less than about 13 mm. In some aspects, radius r′₂ may be less than about 12.75 mm. In some aspects, radius r′₂ may be less than about 12.5 mm. In some aspects, radius r′₂ may be about 12.59 mm. In some aspects, the radius r′₂ may be less (e.g., smaller) than radius r′₃. In some aspects, radius r′₂ may be less than about 80% of r′₃. In some aspects, radius r′₂ may be less than about 85% of r′₃. In some aspects, radius r′₂ may be less than about 90% of r′₃. In some aspects, radius r′₂ may be about 82.7% of r′3.

In some aspects, the sensor or module 2700′ (e.g., an outer perimeter of the sensor or module 2700′) may include a radius r′₃. In some aspects, radius r′₃ may be less than about 15 mm. In some aspects, radius r′₃ may be less than about 15.0 mm. In some aspects, radius r′₃ may be less than about 15.25 mm. In some aspects, radius r′₃ may be less than about 15.5 mm. In some aspects, radius r′₃ may be about 15.22 mm.

FIG. 1Q is a side cutaway view of an example aspect of a sensor or module 2700′. The sensor or module 2700′ includes a barrier construct 2720′, an outer surface 2791′, and a substrate 2716′. The outer surface 2791′ may include light barrier construct portions, lens portions, opaque frame portions, and/or electrode portions. The outer surface 2791′ of the sensor or module 2700′ may face and/or contact the skin of a wearer and may include a generally convex shape. A central region of the sensor or module 2700′ may have a height 2793′. For example, the height of the light barrier construct 2720′ at a central region of the sensor or module 2700′ may correspond to height 2793′. The height 2793′ may be a maximum distance the outer surface 2791′ extends perpendicularly away from the substrate 2716′ (e.g., toward the skin of a wearer). An outer region (e.g., along a perimeter of the substrate 2716′) of the sensor or module 2700′ may have a height 2795′. For example, the height of the light barrier construct 2720′ and/or opaque frame 2726′ at an outer region of the sensor or module 2700′ may correspond to height 2795′. The height 2795′ may be a minimum distance the outer surface 2791′ extends perpendicularly away from the substrate 2716′ (e.g., toward the skin of a wearer).

In some aspects, height 2793′ may be less than about 2.95 mm. In some aspects, height 2793′ may be less than about 2.90 mm. In some aspects, height 2793′ may be less than about 2.85 mm. In some aspects, height 2793′ may be less than about 2.80 mm. In some aspects, height 2793′ may be about 2.85 mm. In some aspects, height 2793′ may be less than about 2.70 mm. In some aspects, height 2793′ may be less than about 2.65 mm. In some aspects, height 2793′ may be less than about 2.60 mm. In some aspects, height 2793′ may be less than about 2.55 mm. In some aspects, height 2793′ may be about 2.58 mm.

In some aspects, height 2795′ may be less than about 1.40 mm. In some aspects, height 2795′ may be less than about 1.35 mm. In some aspects, height 2795′ may be less than about 1.30 mm. In some aspects, height 2795′ may be less than about 1.25 mm. In some aspects, height 2795′ may be about 1.29 mm. In some aspects, height 2795′ may be less than about 1.90 mm. In some aspects, height 2795′ may be less than about 1.85 mm. In some aspects, height 2795′ may be less than about 1.80 mm. In some aspects, height 2795′ may be less than about 1.75 mm. In some aspects, height 2795′ may be about 1.78 mm.

In some aspects, the height 2793′ may be greater (e.g., larger) than height 2795′. In some aspects, height 2793′ may be less than about 230% of height 2795′. In some aspects, height 2793′ may be less than about 225% of height 2795′. In some aspects, height 2793′ may be less than about 220% of height 2795′. In some aspects, height 2793′ may be less than about 215% of height 2795′. In some aspects, height 2793′ may be about 221% of height 2795′. In some aspects, height 2793′ may be less than about 155% of height 2795′. In some aspects, height 2793′ may be less than about 150% of height 2795′. In some aspects, height 2793′ may be less than about 145% of height 2795′. In some aspects, height 2793′ may be less than about 140% of height 2795′. In some aspects, height 2793′ may be about 145% of height 2795′.

Advantageously, a greater height 2793′ (and/or greater ratio of height 2793′ to 2795′) (for example, a taller light barrier at a central region of the sensor or module 2700 may cause light emitted from emitter chambers to travel a greater distance before reaching the detector chambers. Light that travels a greater distance may penetrate deeper into the tissue of the wearer which may improve accuracy of a physiological measurement. A smaller height 2793′ (and/or smaller ratio of height 2793′ to 2795′) may reduce discomfort to the wearer wearing the wearable device 10 or may reduce obstruction to blood flow of the wearer by reducing the amount of pressure the wearable device places on the wearer. The height 2793′ and/or height 2795′ may be selected to balance the above-mentioned considerations such as increasing the depth which light penetrates into the tissue and reducing discomfort or blood flow obstruction of the wearer.

FIG. 1R and FIG. 1S illustrate two example aspects of a sensor or module 2700′ with different light barrier construct configurations. FIGS. 1R and 1S also show an example light path from an emitter chamber to a detector chamber. The light barrier construct 2720′ (or portions thereof) shown in the example aspect of FIG. 1R may be taller (e.g., extending away from a surface of the substrate 2716) and/or wider than the light barrier construct 2720 (or portions thereof) shown in the example aspect of FIG. 1S. The greater height and/or width of the light barrier construct 2720′ in the aspect of FIG. 1R may cause the light emitted from an emitter chamber 2778′ to travel a greater distance before reaching a detector chamber and thus penetrate deeper into the tissue of the wearer than in the aspect of FIG. 1S. Thus, adjusting the height and/or width of the light barrier construct may affect the path the light travels from the emitter chamber to the detector chamber which may affect an accuracy of a physiological measurement. The height and/or width of the light barrier construct may be adjusted, according to various aspects, as required or desired.

FIG. 1T illustrates a cutaway side view of an example sensor or module 2700′ showing light transmissive lens(es) or cover(s) 2702′ and light diffusing material. The light diffusing materials can be included in one or more of the emitter or detector chambers to improve distribution of emitted lighted and/or detected light. The diffusing materials or encapsulant, can include, for example, microspheres or glass microspheres. The encapsulant can eliminate air gaps between the surface of the light transmissive cover 2702′ and the emitters and/or the detectors. The encapsulant can be included around the emitters to more evenly spread the emitted light, causing the emitted light to appear to be emitted from an entire emitter chamber rather than from a point source (that is, a single LED emitter) if the encapsulant were absent. The light transmissive lens(es) or cover(s) 2702′ may include polycarbonate.

FIG. 1U illustrates various embodiments of the device 10. The device 10 can be a MightySat™ fingertip pulse oximeter 802 by Masimo Corporation, which can be worn by the user. The pulse oximeter 802 can measure the user's physiological parameters including, but not limited to, blood oxygen saturation, pulse rate, perfusion index, respiration rate, heart rate, hydration index, glucose, and plethysmographic variability index. The device 10 can be a Radius T™ temperature monitor 804 by Masimo Corporation, which can be worn by the user and can measure the user's temperature. The device 10 can be a W1™ (or Freedom™) smartwatch 806 by Masimo Corporation. The smartwatch 806 can be worn on the user's wrist to non-invasively monitor the user's physiological parameters. The smartwatch 806 can include a display, which can be a touchscreen display. The smartwatch 806 can measure the user's physiological parameters including, but not limited to, blood oxygen saturation, pulse rate, perfusion index, respiration rate, heart rate, hydration index, glucose, and plethysmographic variability index. The device 10 can be a Freedom™ band tracking device 808 by Masimo Corporation, which may be similar to the smartwatch 806 but may not include a display. The device 10 disclosed herein may include physiological sensors, such as Masimo's Radius PCG® sensor, RD Rainbow SET®, Rainbow Acoustic Monitoring® sensors, RD SET® sensors, LNCS® sensors, SofTouch™ sensors, brain monitoring sensors (such as SedLine® and O3® sensors), and specialty sensors, EEG, temperature sensor (skin, core, ambient temperatures), camera, medical or consumer acoustic sensors (e.g., headphones, microphone from a mobile communication device, hearing aid), accelerometer, movement sensor on a mattress or clothes, among other sensors to provide a perspective on the user's sleep activity.

The device 10 can be a Radius PPG™ pulse oximeter 810 by Masimo Corporation, which can be worn by the user. The pulse oximeter 810 can measure the user's physiological parameters including, but not limited to, blood oxygen saturation, pulse rate, perfusion index, respiration rate, heart rate, hydration index, glucose, and plethysmographic variability index. The device 10 can be a Stork™ baby monitor 812 by Masimo Corporation, which can be worn by an infant. The baby monitor 812 can measure the infant's physiological parameters including, but not limited to, blood oxygen saturation, pulse rate, and temperature. The device 10 can be a Centroid™ patient orientation and activity sensor 814 by Masimo Corporation, which can be worn the user. The orientation and activity sensor 814 can track the user's posture, orientation, and activity and measure the user's physiological parameters, such as respiration.

The device 10 can be a blood pressure monitor 816 by Omron Corporation, which can measure the user's blood pressure. The device 10 can be a weight scale 818 by Omron Corporation, which can measure the user's weight. The device 10 can be a TIR-1™ non-contact thermometer 820 by Masimo Corporation, which can measure the user's temperature. Monitoring devices described in this paragraph may not be worn by the user.

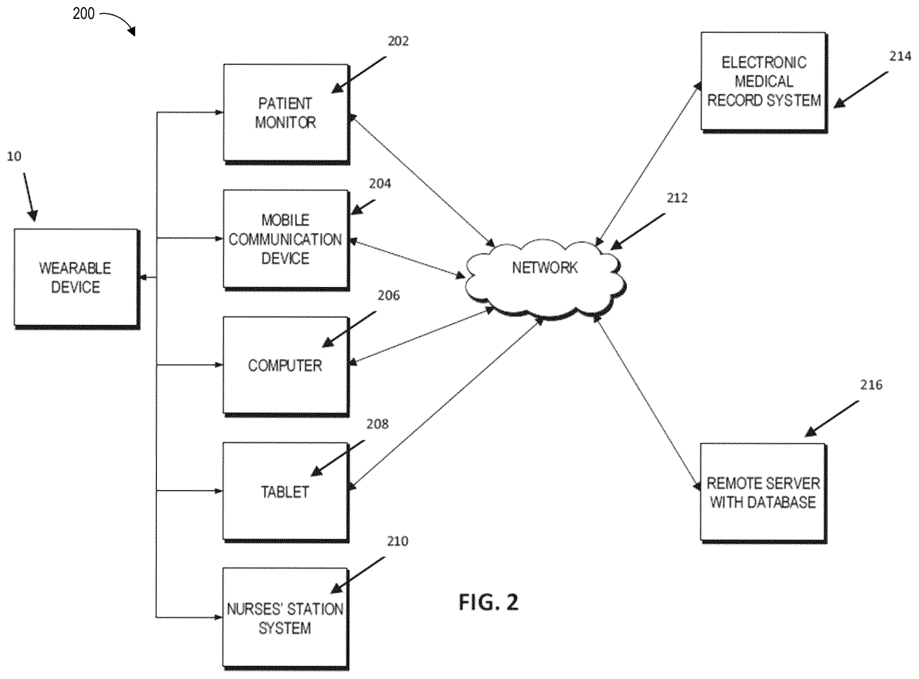

Various different healthcare clouds (e.g., remote server with database 216 in FIG. 2) can be implemented, for instance, based on the location of a user in the world or the type of physiological data being monitored. Identification of a particular healthcare cloud for communicating one or more particular physiological parameters of a particular user can be part of the user's credentials. For example, a location of a healthcare care cloud in the US (such as, the URI or URL) would be part of the user's credentials for a user located in the US. As another example, a location of a healthcare care cloud configured to store a particular type of health data would be part of the user's credentials for the device 10 that collects the particular type of health data.

One or more AV devices located in the user's home can be setup for facilitating monitoring of the user's health using the approaches described herein. When the user is outside the user's home, one or more AV devices located in another premises can be used for monitoring the user's health. For example, the user may be at work, at a home belonging to another person, or the like. The user can setup one or more AV devices located in another premises for uploading physiological data or receiving alerts using the approaches described herein. For instance, the user may be visiting a friend's house. The user's friend can share information for joining the LAN (such as, network name and password). After joining the LAN, the user can setup one or more AV devices located in the friend's house for communicating with the device 10 of the user using the approaches described herein. In some instances, the user's friend can restrict the amount of time the user is permitted to connect to the one or more AV devices. To accomplish this, the AV devices can be configured to communicate with the device 10 of the user for a specified duration of time. For example, the user's friend can utilize an app being executed on the friend's mobile device to inform the AV devices about the limited time duration.

In some cases, other devices can be used in addition to or instead of any of the AV devices. Such other devices can be configured to communicate with the device 10 in order to upload physiological data to the healthcare cloud and receive data from the healthcare cloud. For instance, smart home devices may be used, including, but not limited to, thermostats, cameras, security monitors, refrigerators, washing machines, dryers, remote controls, outlets, hubs, or light switches.

Advantageously, the approaches described herein (such as, in connection with the system 200) can be used for monitoring of the user's health at home and can facilitate early release from a hospital, lessen the need for doctor visits, or the like. Monitoring can be performed in real-time or in substantial real-time using one or more AV devices already present in the user's home or another location, such as another home or work. This can provide redundancy and improve reliability and patient safety. Using the approaches for software or firmware updates described herein, one or more AV devices can be made by a company (or companies) different from that (or those) providing the device 10 or the healthcare cloud.

As described herein, reliable and continuous monitoring of a user's health can be achieved using the device 10 that does not support communication via the LAN (such as, using Wi-Fi). By using a communication protocol that requires lower power consumption, capacity of the power source of a monitoring device can be preserved. This can be important because the device 10 may have limited power source capacity due to being small or inexpensive. Advantageously, prolonged monitoring of the user's health can be achieved. In addition, monitoring can be performed in real-time or substantial in real-time as one or more physiological parameters of the user can be continuously and securely uploaded to the healthcare cloud using one or more AV devices that are already present in the premises where the user is located. Similarly, alerts can be generated and communicated in in real-time or substantial in real-time.

### B. Overview of Continuous Measurement and Intermittent Measurement

Continuous measurement of a user's physiological parameters with a wearable device may assist in identifying a physiological event or other health condition. For example, health conditions can be monitored while the user is asleep. Continuous measurement includes sampling at least one sensor at a periodicity sufficient to consistently capture irregular physiological events while the user is in contact with the wearable device. Continuous measurement may include measuring physiological parameters at a sampling rate until the wearable device identifies a likeliness of a physiological event, and then may increase the sampling rate to better capture physiological parameter data regarding the event. Intermittent measurement is periodically measuring physiological parameters by sampling either by irregular on-demand measurements (spot checking) or at a periodicity that is too slow to consistently capture irregular physiological events (regular checking). Spot checking may include measuring physiological parameters at the instruction of the user. Regular checking may rely on the wearable device to periodically measure physiological measurement, but the intervals may have interruptions where the wearable device may miss important physiological information. Continuous measurements provide the user more information to identify a diagnostic event than with intermittent measurements. For example, continuous measurements may detect second by second change to a user's physiological parameters, such as blood oxygen saturation (SpO2) over the course of their sleep to detect sleep apnea. Intermittent measurements may miss changes in blood oxygen saturation because these changes occur briefly, often within 10 second periods, the time between measurements likely is insufficient to detect the changes. Without continuous measurement, the user may not have the data to identify indicators of sleep apnea. Being slow to diagnose such respiratory events like sleep apnea may expose the user to health risks from low blood oxygen for a duration of time. For example, the user may experience shortness of breath, chest pains, coughing or wheezing, confusion, headaches, rapid heartbeat, and for prolonged lack of treatment, may lead to increased risk of heart disease. Continuous measurement provides the user medically relevant information for assessing whether the user suffers from sleep apnea or other physiological problems.

However, continuously measuring physiological parameters may rapidly drain a wearable device's battery. An approach to continuous monitoring in consumer wearable devices measures the physiological parameters by measuring at an interval of every 5 to 15 minutes when operating in a continuous mode. However, the 5 to 15 minute intervals may be too infrequent to obtain data beneficial to the user for identifying important health changes. For example, sampling the sensors on the wearable device every 5 to 15 minutes may miss medically relevant changes in the user's blood oxygen or other parameters. In general, wearable consumer devices avoid providing continuous monitoring because such measurements require extensive power consumption. Thus, wearable devices currently lack an ability to continuously measure physiological parameters with a wireless wearable device.

The embodiments disclosed herein may provide a system to continuously measure physiological parameters of a user by performing repeated measurements at a series of intervals configured to capture important physiological events. Continuous measurement may include instructing sensors to capture physiological data repeatedly while the user is in contact with the device. For example, optical sensors in a wearable device may emit and detect reflected light from a surface of the user to obtain SpO2 and PR. The optical sensor might include LEDs to emit light at certain wavelengths and detectors to detect the light reflected from the user's skin underneath the optical sensors. The sensors of the wearable device may operate at sampling rate. For example, the sampling rate may include 0 seconds to 1 second, 1 second to 10 seconds, 10 seconds to 20 seconds, or a combination of the disclosed ranges discussed herein. Thus, the embodiments disclosed may provide continuous measurement of physiological parameters.

In response to receiving input about the current state of the physiological parameters, the system may instruct the sensors to capture the physiological data at another sampling rate. Where a second sampling rate may be at a higher or lower frequency than a first sampling rate. The system may monitor for an event as a trigger to increase or decrease the sampling rate. For example, the trigger may correspond to received input from a plurality of sources monitoring the user. In some instances, the plurality of sources may include a PCG, temperature sensor (skin, core, ambient temperatures), posture sensor, accelerometer, pulse oximetry (through motion, low perfusion, optical, or other manner), brain monitoring sensors, EEG, camera, medical or consumer acoustic sensors (e.g., headphones, microphone from a mobile communication device, hearing aid), movement sensor on a mattress or clothes, among other sensors to provide a perspective on the user's activity. When the plurality of sources provide the data indicating a likeliness of a medically-relevant instance occurring, the system may update the sampling rate to more closely monitor the physiological parameters.

### C. Example Hardware Implementations for Measurement Control

Described herein are systems and methods for controlling physiological sensors such as LEDs in a wearable device. Sensor operation can be modulated by changing timing sequences or duty cycles (e.g., time during which they are on and off). Sensors when including LEDs can be further modulated, for example, by changing the intensity or brightness at which they shine. In some aspects, it may be advantageous to modulate LEDs dynamically in real-time based on various conditions, such as a subject's changing physiology or a state of the physiological sensors (e.g., moving, accelerating). For example, LED run times (and/or intensities) can be reduced when additional measurements may not be necessary to improve accuracy, such as when a subject's physiology is static. In some examples, LED run times (and/or intensities) can be reduced when measurements are determined to have a high probability of not being reliable (e.g., noisy signal), such as when the physiological sensors are in motion and/or accelerating. In some examples, the LED run times (and/or intensities) can be increased when additional measurements may be desired to capture and accurately represent a changing physiology of the subject (e.g., changing heart rate, changing blood oxygen saturation, and the like). In some examples, LED run times (and/or intensities) can be increased to obtain more measurement data at periods of high likelihoods of data unreliability or unpredictability, such as when a sensor signal may be noisy due to motion and/or acceleration of the sensor.

Advantageously, modulating LED operation (e.g., updating LED run times and/or intensities at determined times) can advantageously reduce power consumption of the LEDs which can improve system efficiency, operating times, and the like. Moreover, dynamically modulating LED operation based on system conditions (e.g., subject physiology, sensor motion, sensor acceleration, etc.) can reduce power consumption without reducing (or mitigating reduction of) physiological measurement accuracy. Dynamically modulating LED operation can advantageously allow for continuous monitoring of a wearer of a monitoring device, such as for 8 hours of continuous monitoring, 24 hours of continuous monitoring, or more.

Various implementations of the systems and methods disclosed herein may be utilized for continuous measurement of a subject's physiological data and/or to monitor such physiological data in real time. Continuous monitoring can include obtaining physiological parameter data (e.g., one or more signals indicative of a physiological parameter) from one or more sensors (such as any of the sensors disclosed herein) at a sampling rate sufficient to consistently capture irregular physiological events while the user is in contact with the wearable device. Continuous monitoring may include sampling physiological data at a sampling rate until the wearable device identifies a likeliness of a physiological event and then may adjust the sampling rate (e.g., increase or decrease the sampling rate) to better capture physiological parameter data regarding the event. Capturing such changes may be useful for identifying a physiological event or other health condition. Such continuous measurement may allow for monitoring physiological trends and/or for generating a smoother waveform based on the physiological data, which may provide additional information in regard to corresponding physiological parameters. A sampling rate used for continuous measurement may be determined, in some examples, based on a rate at which meaningful changes in physiological data are reasonably anticipated. In some examples, a sampling rate can be defined by relatively short time periods such that even minor changes in the physiological data over time are captured. For example, continuous measurement may include obtaining physiological parameter data at a sampling rate of 1 minute, 30 seconds, 24 seconds, 12 seconds, 11 seconds, 10 seconds, 9 seconds, 8 seconds, 7 seconds, 6 seconds, 5 seconds, 4 seconds, 3 seconds, 2 seconds, 1 second, 0.5 seconds, or 0.1 seconds. A continuous sampling rate may, in some examples, be selected or updated based on anticipated changes in monitored physiological data, sensor type, physiological parameter being monitored or tracked, hardware considerations, or other physiological or hardware considerations.

Monitoring physiological data in real time may include processing, transmitting, and/or displaying such physiological data within a short time period after such data is obtained, for example, within 10 seconds, within 5 seconds, within 4 seconds, within 3 seconds, within 2 seconds, within 1 second, within 0.9 seconds, within 0.7 seconds, within 0.5 seconds, within 0.3 seconds, or within 0.1 seconds from when such data is obtained. Real time monitoring, especially when paired with continuous measurement, can facilitate improved monitoring and treatment, if needed, of physiological conditions that may change in relatively short time periods. This can in turn facilitate accurate detection of changes in physiological parameters and rapid response to such changes (if necessary).

Continuous monitoring of physiological parameters by a wearable monitoring device can be beneficial to the wearer. Continuous monitoring can advantageously improve detection of critical events such as oxygen desaturation events, heart palpitations, or the like. Continuous monitoring can advantageously allow for early detection of physiological abnormalities, such as cardiovascular abnormalities (e.g., arrhythmias, etc.), respiratory abnormalities (e.g., hypoxia, sleep apnea, etc.), or the like. In some examples, health conditions can be monitored while the user is asleep. Continuous monitoring can enable timely medical intervention, which can be crucial in preventing complications or worsening of a physiological condition. Continuous monitoring can lead to improved management of chronic diseases by, for example, keeping track of changes in physiological parameters so that wearers of the wearable device can be proactive in their healthcare routines. Advantageously, continuous monitoring can help to tailor healthcare solutions to individual needs based on, for example, personal physiological data trends.

In some aspects, continuous monitoring of physiological parameters can be achieved by dynamic measurements from a set of physiological sensors. Dynamic measurements may continuously sample physiological data at sampling rates such as described herein. In this way, the dynamic measurements can reduce ordinary power consumption while not missing a significant physiological event (such as, monitoring for sleep apnea throughout the duration of an entire night). Improved battery management, as described herein, can permit a monitoring device to continuously sample physiological data at sampling rates that can permit detection of said physiological events and to continuously sample physiological data at said sampling rates for extended durations. For example, the approaches described herein allow for continuous monitoring of a wearer of the monitoring device, such as for 8 hours of continuous monitoring, 24 hours of continuous monitoring, or more. In some cases, dynamic measurement from the set of physiological sensors can include dynamically modulating one or more LEDs in real-time (as described herein), modulating one or more motion sensors in real-time, and/or modulating other sensors of different types in real-time.

In some aspects, dynamic measurements can include operating LEDs according to an LED mode. An LED mode can include, but is not limited to, a mode of operating one or more LEDs of the wearable device during which a power management condition is applied that may impact the power efficiency of the wearable device. The one or more modes of operation may include, but are not limited to, modes relating to which of a plurality of LEDs and/or other sensors of the wearable device may be turned on or off, be permitted to be turned on or off, have a certain intensity, the like or a combination thereof. The one or more modes may relate to how long and/or when to turn on LEDs having emissions of certain wavelengths and/or wavelength ranges. The one or more modes may relate to how long and/or when to turn on other sensors of different types.

A power management condition may be based on an LED cycle configuration. An LED cycle configuration can include periods of time during which one or more LEDs operate in certain LED modes. In some aspects, when a period corresponding to an LED mode ends (e.g., the controller determines that an end condition is satisfied), the controller may adjust the LED cycle configuration and transition one or more LEDs into a different LED mode of the LED cycle configuration. In some aspects, the controller may determine that one or more LEDs should remain in a certain LED mode. Different LED modes can be associated with different LED run times and/or different LED intensities. In some aspects, the LED cycle configuration can be adjusted in real-time based on various conditions, such as a subject's changing physiology or a state of the physiological sensors (e.g., moving, accelerating). For example, adjusting the LED cycle configuration can include increasing or decreasing a period during which one or more LEDs operate in certain modes. Adjusting the LED cycle configuration can include increasing or decreasing LED run-times associated with certain LED modes. Adjusting the LED cycle configuration can include increasing or decreasing LED intensities associated with certain LED modes.

Described herein are systems and methods for improving physiological measurement accuracy during LED modulation. In some aspects, a system may be configured to not gather physiological data when an LED is turned off. Advantageously, the system described herein can implement post processing routines when LEDs are turned off to maintain physiological measurement output accuracy. For example, when LEDs are turned off and physiological sensors are not collecting data, the system can maintain a buffer of sensor data and/or can interpolate or extrapolate data (e.g., in the buffer). Further described herein are systems and methods for reducing power consumption of a monitoring device without reducing (or mitigating reduction of) physiological measurement accuracy. In some aspects, a system may be configured to gather physiological data from an LED that is on continuously. For example, in certain operating modes, one or more LEDs may be turned off except for at least one LED that remains on. In some aspects, a system may be configured to gather motion data from a motion sensor that is on continuously. For example, in certain operating modes, one or more motion sensors may be turned off except for at least one motion sensor that remains on. In some aspects, motion sensors can include accelerometers, gyroscopes, or the like.

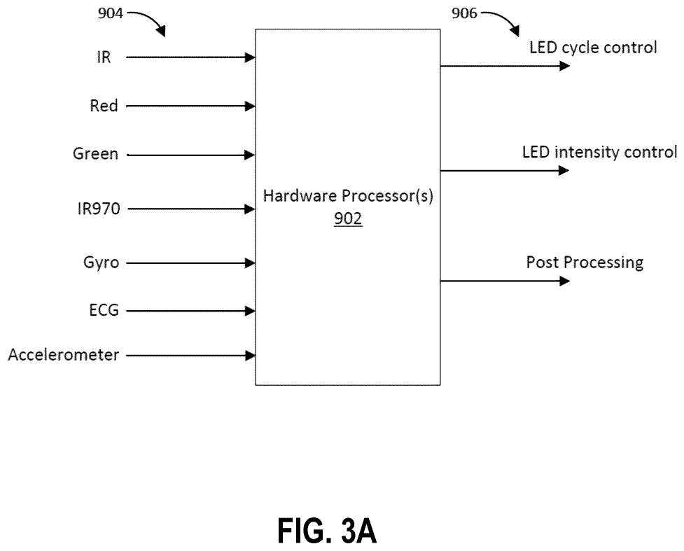

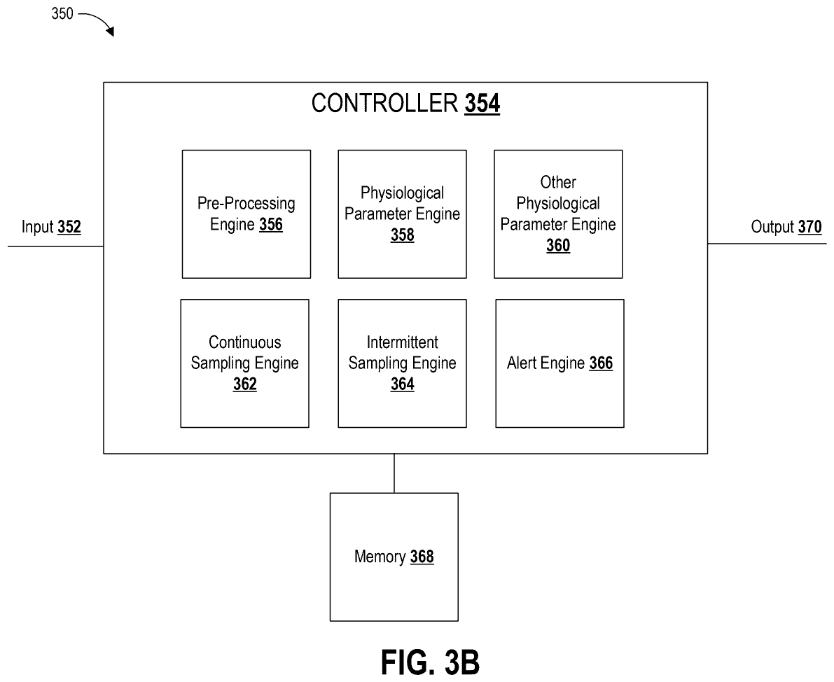

FIG. 3A is a block diagram illustrating example hardware processor(s) 902 implemented on a sensor device, such as a wearable device. The hardware processor(s) 902 can be configured to receive and process one or more input signals and generate and output one or more output signals.

The hardware processor(s) 902 can receive sensor data 904. Sensor data 904 may be raw (e.g., unprocessed, unfiltered, etc.) sensor data. In some aspects, sensor data 904 may correspond to input signals. The input signals may include but are not limited to one or more signals from one or more sensors, such as optical sensors, motion-based sensors, or other types of sensors. Sensor data 904 may be processed and/or filtered data such as processed parameter values. In some aspects, sensor data 904 may include output values, which may include, but is not limited to one or more physiological parameters or processed signals. In some examples, one or more physiological parameters may include one or more of an SpO₂ value, pulse rate (PR), perfusion index (PI), pulse variability index (PVI), respiration rate (RRp), ECG, and hydration index, blood oxygen, heart rate (HR), skin temperature, core temperature, ambient temperature, movement, body position, respiration rate, heart rate variability (HRV), pulse rate variability (PRV), electrocardiogram (ECG), acoustic data, and/or the like.

The hardware processor(s) 902 can process the sensor data 904 to generate output controls 906. The output controls 906 can include one or more signals to control operation of one or more sensors, for example, to control cycle timing of the LEDs and/or strength or intensity of the LEDs. The output controls 906 can include one or more signals to control post processing of physiological data.

As illustrated in FIG. 3B, in some examples, a system 350 may include a controller 354, such as illustrated in FIG. 3B, to perform one or more processes relating to monitoring physiological parameters as described herein. The controller 354 may be included, for example, by one or more hardware processors (for example, hardware processor(s) 902 as described in FIG. 3A) associated with a system including a physiological monitoring device, such as a wearable device described herein. For example, the one or more hardware processors may be associated with local and/or remote computing systems, such as referenced herein with reference to FIG. 2. In some examples, the wearable device (e.g., 10 in FIG. 2) and/or the remote server with database (e.g., database 216 in FIG. 2) can include the controller 354. In some examples, the controller 354 may be configured to compute methods and processes in real-time. In this manner, the controller 354 can include hardware capabilities for real-time processing.

An example controller 354 may include one or more processes or engines configured to process input 352 to generate an output 370. The one or more processes or engines may include, but are not limited to a pre-processing engine 356, physiological parameter engine 358, other physiological parameter engine 360, continuous sampling engine 362, intermittent sampling engine 364, and alert engine 366. However, the number and types of engines in FIG. 3B is given as an example and is not meant to be limiting.

In at least one example, an input 352 may include, for example, one or more physiological signals from one or more physiological monitors configured to measure data from a user, and/or a data stream for physiological data (e.g., SpO2, PR, RRp, resting PR, peripheral arterial tonometry (PAT)). Other or fewer parameters may also be used. For example, in some examples, temperature, sweat, stress, or other parameter data stream may be taken as input by the controller 354. In some examples, the input 352 may be user input (e.g., data regarding the user's age, demographic, questionnaire answers, etc.), physiological sensor inputs, such as photoplethysmography (PPG), accelerometer data, temperature data, acoustic data, and/or other physiological signals (or any combination of physiological signals). In some instances, the controller 354 may retrieve (for example, continuously or intermittently) raw data as input 352 from the memory 368.

In at least one example, an output 370 may include an alert to the user regarding the occurrence of a physiological event. For example, the output 370 may provide the user an alert on a user interface (UI) on the wearable device, mobile communication system, web-based application, or another suitable interface to the user. In some instances, the output may provide the user their physiological parameters measured by the wearable device, external sensors, an assessment from the physiological parameters, or other indicators that may be used for providing an alert regarding the occurrence of the physiological event.

To monitor physiological parameters, the controller 354 may pre-process the input (e.g., input 352) to obtain enhanced signal quality for continuously monitoring physiological parameters of the user and provide the pre-processed signal to engines disclosed herein (e.g., the physiological parameter engine 358 and/or the other physiological parameter engine 360). To achieve the pre-processing, the controller 354 may include a pre-processing engine 356 to perform the pre-processing of the input. The input may include raw sensor signals obtained from at least one sensor.

The controller 354 may be configured to monitor physiological parameters, such as those disclosed herein. In some examples, the controller 354 may include a physiological parameter engine 358 to achieve the function of determining SpO2 of the user. The physiological parameter engine 358 may obtain a pre-processed signal from the pre-processing engine 356 to determine the SpO2 of the user. The physiological parameter engine 358 may provide physiological parameter computation to the continuous sampling engine 362 as continuous measurements.

The controller 354 may be configured to monitor physiological parameters, such as those different from SpO2 and PR. In some examples, the controller 354 may include an other physiological parameter engine 360 to achieve the function of determining other physiological parameters of the user. The other physiological parameter engine 360 may provide physiological parameter computation to the continuous sampling engine 362 as continuous measurements. The other physiological parameter engine 360 can be configured to obtain the physiological parameter data based on measured information. For example, the other physiological parameter engine 360 can be configured to obtain the physiological parameter data from various sources of information (e.g., from sensors described herein).

The controller 354 may be configured to continuously monitor physiological parameters for determining a physiological event. In some examples, the controller 354 may include a continuous sampling engine 362 to continuously measure the physiological parameters of the user. The continuous sampling engine 362 may provide the continuous measurement, or an alert signal, to the alert engine 366.

The controller 354 may be configured to intermittently monitor physiological parameters for determining a physiological event. In some examples, the controller 354 may include an intermittent sampling engine 364 to periodically measure the physiological parameters of the user. The intermittent sampling engine 364 may provide the intermittent measurements, or an alert signal, to the alert engine 366.

The controller 354 may be configured to alert the user of an occurrence of the physiological event. In some examples, the controller 354 may include an alert engine 366 to alert the user when the physiological event occurs. The alert engine 366 may obtain an alert signal from engines disclosed herein (e.g., the continuous sampling engine 362 and the intermittent sampling engine 364). In some cases, the alert engine 366 receives the alert signal in response to the physiological event occurring.

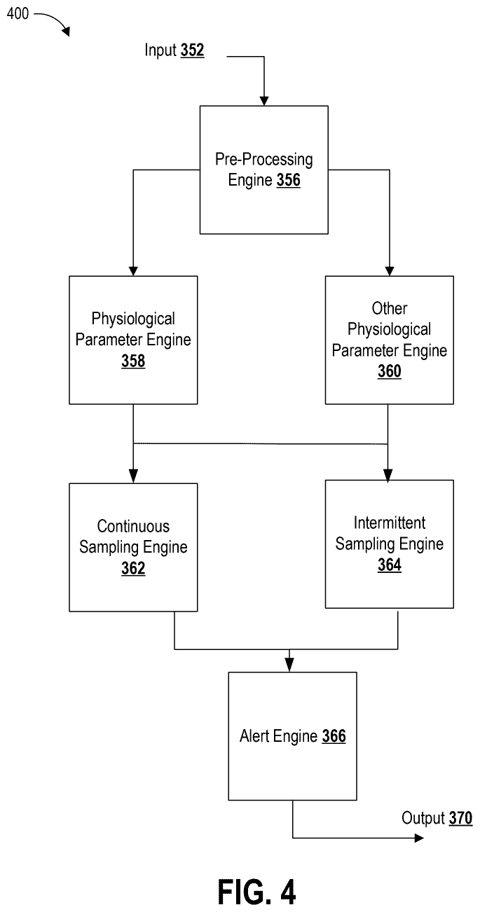

FIGS. 4-6 illustrate various example embodiments (including an exemplary system 400 of operation for the controller 354), processes, and outputs of the controller 354 configured to process input 352 to generate an output 370, such as an alert indicator, user information, physiological parameters, control signal, and/or display information.

### D. Example Pre-Processing Engine

To monitor physiological parameters, the controller 354 may pre-process the input (e.g., input 352) to obtain enhanced signal quality for continuously monitoring physiological parameters of the user and provide the pre-processed signal to engines disclosed herein (e.g., the physiological parameter engine 358 and/or the other physiological parameter engine 360). The input may include raw sensor signals obtained from at least one sensor. The controller 354 may include a pre-processing engine 356 to perform the pre-processing of the input. The preprocessing of the input may include filtering to remove noise caused by interference (e.g., motion by the user, ambient light). The pre-processing engine 356 may implement a combination of physical design (e.g., ensuring the sensor fits snugly against the skin) and digital filtering techniques to minimize interference. For example, the pre-processing engine 356 may compute algorithms such as adaptive filtering for mitigating noise by adjusting parameters based on detected motion levels. The motion levels may correspond to movement data from an accelerometer. In some cases, the pre-processing engine 356 may mitigate ambient light interference. Light entering the sensor from external sources can alter the signal. The pre-processing engine 356 may mitigate electrical interference. For example, the pre-processing engine 356 may include shielding and a circuit design that reduces noise from electrical sources. The pre-processing engine 356 may apply a low-pass filter to the signal to obtain a filtered signal. In some examples, the pre-processing engine 356 may eliminate noise by applying the low-pass filter. For example, the low-pass filter may have an upper frequency band to cut off frequencies above the upper band. In some cases, the upper band may correspond to noise from a user's physiology and movement. In some examples, the pre-processing engine can be configured to apply a FIR filter. The FIR filter can include a low-pass filter. The low-pass filter frequency can be any number, for example, 8 Hz. In some instances, the pre-processing engine 356 may apply a high-pass filter. The high-pass filter may have a lower frequency band to cut off frequencies below the lower frequency band. In some cases, the lower frequency band may correspond to noise such as the ambient light interference. The pre-processing engine 356 may perform a baseline wander removal. Baseline wander, or slow-moving trends in the at least one sensor signal, can obscure a true pulsatile waveform. The pre-processing engine 356 may filter, using a high-pass filter, to remove the slow-moving trends without affecting the integrity of the pulse signal.

The pre-processing engine 356 may downsample data received from the input. In some examples, down sampling can provide an increased ability for the pre-processing engine to perform computational signal processing. In some examples, the pre-processing engine can down sample the input from a first frequency to a second frequency. For example, the pre-processing engine may downsample the input from 2000 Hz to 62.5 Hz, from 62.5 Hz to 1 Hz, or another downsampling process applicable for continuous measuring of physiological parameters.

The pre-processing engine 356 may divide the input into one or more windows of time. For example, the window may correspond with a periodic interval during which the at least one sensor measures physiological data. The pre-processing engine 356 may shift the window along the input according to a time axis windowed with the periodic interval. As the window is shifted, there may be an overlap of time between windows such that both a first window and a second window may contain an overlapping period of time. To perform windowing, the pre-processing engine 356 may multiply the input by a window function. In some examples, a short window may correspond to a high time and low frequency resolution, whereas a long window may correspond to a low time and a high frequency resolution.

The pre-processing engine 356 may adjust a sampling rate to obtain data from the input. The sampling rate may be at a rate to capture medically relevant details of the input from the at least one sensor. In general, the rate is at least twice the highest frequency component of the signal, according to the Nyquist theorem. The pre-processing engine 356 may operate at a first sampling rate corresponding to continuously monitoring physiological parameters of the user, and update to a second sampling rate when a likeliness of a physiological event is above threshold. The first sampling rate may be 0.01 Hz, 0.02 Hz, 0.05 Hz, 0.1 Hz, 0.15 Hz, 0.5 Hz, 1 Hz. The second sampling rate may be increased or decreased from the first sampling rate. The pre-processing engine 356 may obtain a signal to update the sampling rate from the first sampling rate to the second sampling rate when a triggering event occurs. The triggering event may include a medically relevant event, such as identifying a likeliness of a physiological event.

The pre-processing engine 356 may perform signal normalization. Normalizing the signal can account for variations in signal strength due to differences in skin thickness, pigmentation, and sensor placement. The pre-processing engine 356 may adjust the signal amplitude to a standard range, facilitating more consistent analysis. In some instances, the pre-processing engine 356 may increase a power to the at least one sensor to increase the amplitude of the signal. In some cases, the increased amplitude may correspond with the input having enhanced sensor sensitivity (e.g., physiological parameter data having increased numerical resolution). In some cases, the pre-processing engine 356 may decrease sensitivity of the at least one sensor. The pre-processing engine 356 may perform dynamic range adjustment. Adjusting the dynamic range of the signal to match the input range of the analog-to-digital converter (ADC) enhances the resolution of the signal digitization process, improving the quality of the data for analysis. The pre-processing engine 356 may perform calibration. The pre-processing engine 356 may calibrate against known standards to adjust operation for systematic biases in the measurement process. For example, the pre-processing engine 356 may adjust depending on a pigment of the user's skin.

The pre-processing engine 356 may perform artifact detection and rejection. Identifying segments of the at least one sensor signal that are corrupted for reliable analysis may provide for increased quality signal. The pre-processing engine 356 may set thresholds for signal quality based on an amplitude and frequency of an input waveform and rejecting or flagging data that fails to meet these criteria.

The pre-processing engine 356 may perform temperature compensation. In some instances, the at least one sensor signal can be affected by a temperature of the skin or core of the user or an ambient temperature of an environment. The pre-processing engine 356 may increase or decrease sensitivity of the at least one sensor to measure the input according to a temperature measurement. For example, when the temperature is above a temperature threshold, the pre-processing engine 356 may decrease sensitivity (or increase sensitivity). When the temperature is below a temperature threshold, the pre-processing engine 356 may increase sensitivity (or decrease sensitivity).

The pre-processing engine 356 may perform timing control of the at least one sensor. For example, the pre-processing engine 356 may control a timing of LED activation of a PPG sensor, ensuring that readings adjust according to the skin or core temperature of the user or the ambient temperature. For example, when the temperature is above a temperature threshold, the pre-processing engine 356 may decrease (or increase) the timing of LED activation. When the temperature is below a temperature threshold, the pre-processing engine 356 may increase (or decrease) the timing of LED activation.

### E. Example Physiological Parameter Engine

The controller 354 may be configured to continuously monitor physiological parameters, such as PR and blood oxygen saturation (SpO2). In some examples, the controller 354 may include a physiological parameter engine 358 to obtain PR and SpO2 of the user. The physiological parameter engine 358 may obtain a pre-processed signal from the pre-processing engine 356 to determine the PR and/or the SpO2 of the user. The physiological parameter engine 358 may provide physiological parameter computation to the continuous sampling engine 362 as continuous measurements. In some instances, the physiological parameter engine 358 may identify pulse peaks and troughs from the pre-processed signal. The pre-processed signal may be analyzed to identify systolic peaks (highest points corresponding to heartbeats) and diastolic troughs (lowest points). The physiological parameter engine 358 may compute algorithms, such as peak detection algorithms that identify local maxima and minima in the pre-processed signal, for identifying the peaks.

The physiological parameter engine 358 may calculate absorption ratios corresponding to a level of absorption of transmitted light by the user's skin. In some instances, the physiological parameter engine 358 may compute the absorption of light at the red and infrared wavelengths for each detected pulse. The physiological parameter engine 358 may measure an amplitude of the pre-processed signal (difference between peak and trough values) at both wavelengths. The physiological parameter engine 358 may compute the ratio between a transmitted signal to a received signal from both wavelengths to obtain the absorption ratios for each wavelength.

The physiological parameter engine 358 may perform Ratio of Ratios (ROR) calculation. The ROR is a dimensionless number that is used to estimate SpO2 levels. In some instances, the physiological parameter engine 358 may compute the ratio of the red to infrared signal amplitudes (which corresponds to the absorption at these wavelengths) for each pulse. The physiological parameter engine 358 may average the ROR over several pulses to reduce the impact of transient noise or motion artifacts.

The physiological parameter engine 358 may compute an SpO2 calculation. The physiological parameter engine 358 may determine an empirical relationship between the ROR and SpO2 levels (typically nonlinear relationship). The physiological parameter engine 358 may use calibration curves based on clinical research to map the ROR to specific SpO2 values. The physiological parameter engine 358 may calibrate the curve to map the ROR to SpO2 values with a look-up table or mathematical model to translate the ROR into an SpO2 percentage. In some instances, the physiological parameter engine 358 may apply a calibration curve or equation to compute the SpO2 value. The physiological parameter engine 358 may estimate the SpO2 level with a calculated ROR as applied to the calibration curve or from a mathematical equation. The curve or equation used may follow principles based on the Beer-Lambert law. In some instances, the physiological parameter engine 358 may adjust for individual variability. The physiological parameter engine 358 may compute algorithms to adjust the estimated SpO2 based on factors that can influence accuracy, such as skin pigmentation, thickness, and temperature.

The physiological parameter engine 358 may compare the SpO2 value to a Signal Quality Index (SQI). If the signal quality is poor (e.g., due to motion artifacts), the physiological parameter engine 358 may flag the reading as unreliable or attempt to re-acquire the data.

The physiological parameter engine 358 can be configured to obtain the PR based on measured cardiac information. For example, the physiological parameter engine 358 can be configured to obtain the PR from various sources of information (e.g., an acoustic system, PPG, PR monitoring system, ECG system, among various other sources). In some examples, the physiological parameter engine 358 can be configured to compute PR information in real-time. In this manner, the physiological parameter engine 358 can include hardware capabilities for real-time processing. In some examples, the physiological parameter engine 358 can perform the methods disclosed herein from stored data (for example, from the memory 368).

### F. Example Other Physiological Parameter Engine

The controller 354 may be configured to continuously monitor physiological parameters. In some examples, the controller 354 may include an other physiological parameter engine 360 to achieve the function of measuring physiological parameters of the user. The other physiological parameter engine 360 may provide physiological data to the continuous sampling engine 362 as continuous measurements. In some cases, the other physiological parameters are components of the input from the at least one sensor that are different from the physiological parameter engine 358. In some instances, the sensors collecting the other physiological parameter data are included in the wearable device. In some instances, the sensors are external to the wearable device. Other physiological parameters may include physiological parameters and sensors as disclosed herein.

In some examples, the other physiological parameter engine 360 may obtain data from sensors included in the wearable device and/or external from the wearable device. For example, the other physiological parameter engine 360 may obtain acoustic data from a mobile communication device placed near the user and recording the user while asleep. In this case, the mobile communication device may provide acoustic data when the user is asleep, such as breathing sounds, snoring, or breathing patterns indicating a risk for a physiological event (such as the user's lack of breathing possibly caused by an obstruction). The mobile communication device may provide the acoustic data to the other physiological parameter engine 360. The other physiological parameter engine 360 may process the acoustic data and transmit the processed acoustic data to the continuous sampling engine 362.

In some examples, the other physiological parameter engine 360 may obtain the user's body position and temperature (skin, core, and ambient). Some positions of the user's body may be more prone to a physiological event than other positions (lying on the user's back is more likely to cause a respiratory event than on their side). In this case, the other physiological parameter engine 360 may obtain accelerometer data from the wearable device to detect a position of the user's body while sleeping. The other physiological parameter engine 360 may obtain a temperature of the user's skin, core, or an ambient temperature of the environment. The other physiological parameter engine 360 may process the accelerometer data and the temperature data and transmit the processed data to the continuous sampling engine 362.

In some examples, the other physiological parameter engine 360 may obtain the user's ECG signal. In some cases, the ECG signal may be used to identify whether the user may be diagnosed with atrial fibrillation according to irregularities in the user's cardiac electrical activity. The other physiological parameter engine 360 may process the ECG data and transmit the processed data to the continuous sampling engine 362.

### G. Example Continuous Sampling Engine

The controller 354 may be configured to continuously monitor physiological parameters. Continuously monitoring the physiological parameters may assist in identifying a likeliness of a physiological event. The physiological event may include changes of physiological parameters impacting the user's health conditions, changes of physiological parameters used for diagnostic purposes, and/or user-specific conditions. The user-specific conditions may include respiratory events (such as respiratory depression, obstruction, or cessation of breathing), cardiac events (such as atrial fibrillation), sleep quality tracking, stress tracking, and/or other physiological parameter monitoring as disclosed herein. In some examples, the controller 354 may include a continuous sampling engine 362 to continuously measure the physiological parameters of the user. The continuous sampling engine 362 may provide the continuous measurement or an alert signal to the alert engine 366.

In some instances, the continuous sampling engine 362 may perform data collection and storage. For example, the continuous sampling engine 362 may collect physiological parameter data continuously, such as SpO2 readings, from at least one sensor (e.g., via the physiological parameter engine 358) at regular intervals corresponding to a sampling rate. In some cases, the continuous sampling engine 362 may timestamp the data received at the regular intervals. In some instances, the continuous sampling engine 362 may store the data in a database. The continuous sampling engine 362 may store the data in local or remote storage. For example, the continuous sampling engine 362 may store the data in the memory 368. In some cases, the continuous sampling engine 362 may store the data in remote server with a database (e.g., remote server with database 216 in FIG. 2).

The continuous sampling engine 362 may perform a trend analysis on the physiological parameter data obtained from the at least one sensor (e.g., via the physiological parameter engine 358 and/or other physiological parameter engine 360). In some instances, the continuous sampling engine 362 may continuously monitor the physiological parameter levels from the data over time to identify trends (e.g., drops in oxygen saturation during sleep). In some instances, the continuous sampling engine 362 may implement a moving average calculation by applying moving averages or other smoothing techniques to the physiological parameter data to identify underlying trends while mitigating short-term fluctuations. For example, the continuous sampling engine 362 may implement a moving average filter in software that calculates the average physiological parameter value over a configurable window of time (e.g., the last 10 readings) to smooth out short-term fluctuations. In some instances, the continuous sampling engine 362 may perform the trend analysis with trend detection algorithms. The trends may include a gradual decrease in physiological parameter levels over time, using statistical methods. For example, the statistical methods for the applications disclosed herein may include linear regression techniques or machine learning models. In some cases, the linear regression techniques may include weights assigned to a plurality of features to identify physiological trends. The machine learning model can be configured to apply classifiers. For example, the classifiers can include support vector machine (SVM), twin support vector machine (TWSVM), Gaussian mixture models, random forests, artificial neural networks (ANNs), CNNs, recurrent neural networks (RNNs), deep neural networks (DNNs), generative adversary networks (GANs), large language models (LLMs), and/or transformers (e.g., generative pretrained transformers (GPT) and not pretrained transformers), or another form of machine learning classifier.

The continuous sampling engine 362 may also perform a variability analysis. For example, the continuous sampling engine 362 may analyze the variability in physiological parameter readings, identifying periods of high fluctuation or variability that might indicate unstable health conditions. In some instances, the continuous sampling engine 362 may calculate statistical measures for the processes disclosed herein, such as standard deviation or variance on the physiological parameter data over time to assess data variability.

In some instances, the continuous sampling engine 362 may operate at a sampling rate to obtain physiological parameter data from the user. In some cases, the sampling rate may be predetermined based on a data freshness standard. For example, the data freshness standard may be between 0 to 1 second, 1 to 10 seconds, 10 to 20 seconds, 10 to 30 seconds, or a combination of the ranges as disclosed herein. In some cases, having a sampling rate lower than 1 Hz increases a risk of missing clinically relevant events. Parameters may change second to second during a physiological event. For example, sampling at 0.5 Hz, the measurements obtained by the continuous sampling engine 362 begin to blend event details together. During a physiological event, such as a respiratory event, a rate of oxygen desaturation at 0.5 Hz may characterize the respiratory event effectively, but may have difficulty to determine the SpO2 data for individual events. Sampling rates any slower may entirely miss the changes in SpO2 data altogether. In some cases, when interpolating data from a lower sampling rate to a faster rate (for example, from a 0.1 Hz sampling interpolated to 1 Hz sampling), the information obtained from the interpolation may be different than if the continuous sampling engine 362 were to actually sample at 1 Hz. Thus, the continuous sampling engine 362 may operate at a single sampling rate, such as 1 Hz.

The continuous sampling engine 362 may compare data from at least one of the physiological parameters to a threshold (such as, a parameter threshold). In some instances, the threshold may correspond to known ranges of physiological values. The known ranges of the physiological values may be derived from objective medical resources in identifying health conditions of a user. The threshold may correspond to a baseline. The baseline may correspond to a user baseline. The baseline may be predetermined, pre-programmed, or determined from the user's normal use of the wearable device. For example, the unique baseline to the user may be derived from averaging physiological parameter data collected by the wearable device for a duration of use by the user (e.g., 0-24 hours, 24-48 hours, 48-72 hours, 72-96 hours, 96-120 hours, 120-144 hours, 144-168 hours, greater than 168 hours, or any intermediate range of the ranges disclosed herein). In some instances, the threshold may correspond to a standard. In some cases, the standard is a clinical standard. For example, the continuous sampling engine 362 may compare the physiological data to the clinical standard including a model of clinical stability. The clinical standard may include typical limits known by medical personnel. For example, the continuous sampling engine 362 may consider both low and high extremes that may require attention. The low and high threshold may correspond with the baseline and/or objective standards. For example, the continuous sampling engine 362 may establish SpO2 thresholds (e.g., below 90% for a specified period) based on clinical guidelines. The physiological parameter values may be configurable to allow for updates based on new research, user health condition, medical personnel (or user) preference, or user customization (high, medium, or low sensitivity). In some instances, the threshold may include a deviation of the physiological data. The deviation may be a value indicative of a stable health of the user. For example, when measuring SpO2 data, the continuous sampling engine 362 may measure a change in the SpO2 data greater than a standard deviation of the data values. In some cases, the deviation may be a percentage from the standard deviation, standard, known ranges, and/or baseline, such as greater than a 3% deviation.

In some examples, the continuous sampling engine 362 may determine a movement condition of the wearable system based at least in part on data from at least one motion sensor. The movement condition may include an elevated movement or reduced movement.

In some examples, the continuous sampling engine 362 may determine a physiological stability of the wearer based at least in part on at least one physiological signal, the physiological stability comprising a stable condition, an unstable condition, and/or the like. In some cases, the at least one physiological signal may be associated with the light after attenuation by tissue corresponding to a wrist or a finger of the wearer. The at least one physiological signal may be associated with light within the first wavelength range after attenuation by tissue of the wearer. In some cases, determining whether the wearer has a stable physiological condition or an unstable physiological condition may include the continuous sampling engine 362 determining whether a physiological threshold condition is satisfied.

In some examples, the continuous sampling engine 362 may select a mode of operating a sensor as disclosed herein (for example, operating a plurality of LEDs for an optical sensor). The selection of the mode of operating the sensor may based at least in part on the movement condition and the physiological stability of the wearer. The physiological threshold condition may be based in part on a history of physiological data received from one or more detectors. In some cases, determining whether the physiological threshold condition is satisfied may include the continuous sampling engine 362 determining whether a value, a change, a rate of change, or a change in the rate of change of the physiological data satisfies the physiological threshold condition.

In some cases, a first mode of operation may be associated with elevated movement or unstable physiological condition, where the first mode of operation may include a first power management condition comprising permitting the plurality of LEDs to emit light. The determination of whether the movement condition is elevated or reduced may include the continuous sampling engine 362 determining whether a movement threshold condition is satisfied. The movement threshold condition may be based in part on a history of movement data received from the at least one motion sensor. In some examples, determining whether the movement threshold condition is satisfied may include the continuous sampling engine 362 determining whether a value, a change, a rate of change, or a change in the rate of change of the movement data satisfies the movement threshold condition.

In some cases, the first power management condition may include permitting all LEDs of the plurality of LEDs to emit light. The first power management condition may include permitting to emit light a first set of the plurality of LEDs that are configured to emit light within a first wavelength range and disallowing from emitting light a second set of the plurality of LEDs that are configured to emit light within a second wavelength range (where the wavelength ranges disclosed herein can be any infrared, visible light, and/or ultraviolet wavelengths, or any range outside these examples). In some cases, the first power management condition may include turning on additional motion sensors configured to detect motion of the wearable system.

In some cases, a second mode of operation may be associated with reduced movement and stable physiological condition, where the second mode of operation may include a second power management condition comprising permitting to emit light a first set of the plurality of LEDs that are configured to emit light within a first wavelength range and disallowing from emitting light a second set of the plurality of LEDs that are configured to emit light in a second wavelength range. In some cases, the first set may be different from the second set. Disallowing the second set from emitting light may be based on hardware considerations or physiological considerations. In some examples, when operating in the second mode, the continuous sampling engine 362 may maintain a buffer of physiological data associated with light within the second wavelength range after attenuation by tissue of the wearer.

In some cases, a third mode of operation may be associated with reduced movement and stable physiological condition. The third mode of operation may include a third power management condition that disallows emitting light from the plurality of LEDs. The third power management condition may include turning off the at least one motion sensor.

In some examples, the continuous sampling engine 362 may adjust operation of the plurality of LEDs based on the selected mode. The adjusting the operation of the plurality of LEDs may include increasing a period of the first mode or decreasing a period of the first mode, or increasing a period of the second mode or decreasing a period of the second mode.

In some instances, the continuous sampling engine 362 may compare changes in correlated physiological parameters. In some cases, the physiological parameters as disclosed herein may each be correlated to one another. The presence of the physiological event may be shown by changes in a number of physiological parameters. For example, in a situation where the user is experiencing the physiological event (such as a respiratory event), the user's SpO2 may change (e.g., may decrease in value over time), the user's PR (measured from PPG, or HR if measured from ECG) may change (e.g., may increase in value over the same time interval), the user's PI may change, the user's RRp may change (e.g., may decrease over time), the user's PRV (measured from PPG, or HRV if measured from ECG) may change (e.g., increase in PRV over time), the user's movement may change (e.g., accelerometer may indicate the user increases in movement), the user's body position may correlate with an increased likelihood of the respiratory event (e.g., most obstructions occur when the person sleeps on their back, so having some indication of when the person is on their back may be a trigger to increase sampling), the user's PAT may change (e.g., thoracic pressure may change as the user cannot breath), and/or the user's breathing sound may change (e.g., no breathing sounds when the respiratory event occurs). In some cases, the continuous sampling engine 362 may measure the user's RRp to indicate a normal breathing activity of the user or a lack of the physiological event. Thus, by comparing changes in correlated physiological parameters, the continuous sampling engine 362 may identify the physiological event.

In some instances, the continuous sampling engine 362 may estimate the likelihood of the physiological event including quantifiable percentages, confidence scores and/or intervals, qualitative categories, and/or a combination of the foregoing. In some cases, the continuous sampling engine 362 may compute a confidence score to obtain a probability of likelihood of the physiological event. The confidence score may correspond with a standard deviation of a known value, standard, and/or baseline with respect to the physiological parameters. For example, the standard deviation from the known value, standard, and/or baseline may include a 95% confidence interval (95% CI). The known value, standard, and/or baseline may be determined as disclosed herein. In some examples, the continuous sampling engine 362 may compare a deviation of the SpO2 (e.g., a decrease in SpO2) with a deviation in PR (e.g., an increase in PR) to detect the occurrence of the physiological event. In some cases, the amount of deviation for SpO2 may be outside an SpO2 baseline confidence interval (e.g., 95% CI) and the amount of deviation for PR may also be outside a PR baseline confidence interval (e.g., 95% CI). In this case, the continuous sampling engine 362 may compute a probability indicating the physiological event is likely, which may trigger generation of an alert. In some examples, the continuous sampling engine 362 may combine the deviations for each respective physiological parameter and compare the result to a predetermined threshold. When the combination of the deviation is above the predetermined threshold, the continuous sampling engine 362 may determine the physiological event is likely. In some instances, the likelihood may correspond to a qualitative determination between values of blood oxygen data and values of PR data. In this case, the likelihood of the physiological event may be above the predetermined threshold when a drop in the values of the blood oxygen data occurs when an increase in the values of the PR data occurs. Although this example correlates SpO2 and PR, other correlations may exist between the physiological parameters identified herein. Thus, the continuous sampling engine 362 may determine the likelihood of the physiological event from sources of data external from the wearable device.

In some instances, the continuous sampling engine 362 may compute a likeliness of a physiological event from various sources of physiological parameter data. The continuous sampling engine 362 may obtain the physiological parameter data from sensors on a wearable device (e.g., via the physiological parameter engine 358 and/or the other physiological parameter engine 360) and/or from sensors external to the wearable device. The sensors external to the wearable device may be situated in the room where the user occupies. The sensors external to the wearable device may include such sensors as disclosed herein. In some examples, the continuous sampling engine 362 may obtain acoustic data from a microphone (for example, of a mobile communication device placed near the user while asleep) to obtain sounds (such as, respiration sounds). In this case, the mobile communication device may provide acoustic information about the user's sleep, such as breathing sounds, snoring, or breathing patterns indicating a risk for a physiological event (such as the user's lack of breathing possibly caused by an obstruction). The mobile communication device may provide the acoustic data to the continuous sampling engine 362. The continuous sampling engine 362 may, for example, identify a duration of the lack of breathing and classify the duration as a physiological event. For example, the continuous sampling engine 362 may include classification engines to identify correlations from the various sources of data to classify events, such as the lack of breathing as a physiological event (e.g., sleep apnea). In this case, the lack of breathing may correlate and be evident according to another physiological parameter, for example, SpO2 data. In some examples, the SpO2 data may show a decreasing trend when the user stops breathing because the lack of oxygen flowing corresponds with a decrease in measurable oxygen in the user's body. Although the example correlates acoustic data and the user's breathing, other correlations may exist to determining a likeliness of the physiological event. For example, the user's body position and temperature (skin, core, and ambient) may correlate to detect a physiological event. Some positions of the user's body may be more prone to a physiological event than other positions (lying on the user's back is more likely to cause a respiratory event than on their side). In this case, the continuous sampling engine 362 may obtain accelerometer data from the wearable device to obtain body position data of the user's body (for example, while sleeping). The continuous sampling engine 362 may obtain a temperature of the user's skin, core, or an ambient temperature of the environment. Thus, the continuous sampling engine 362 may compute the likeliness of the physiological event from various sources of physiological parameter data, including sensors on a wearable device (e.g., via the physiological parameter engine 358 and/or the other physiological parameter engine 360) and/or from sensors external to the wearable device.

In some examples, the continuous sampling engine 362 may update a sampling rate of the at least one sensor. The continuous sampling engine 362 may update the sampling rate from a first sampling rate to a second sampling rate (such as, a new sampling rate). In some instances, the first sampling rate may be lower than the second sampling rate. In some cases, the first sampling rate may be higher than the second sampling rate. In some cases, the continuous sampling engine 362 may update the sampling rate in response to determining a likelihood of a physiological event. For example, when the continuous sampling engine 362 determines the user likely experienced a physiological event, such as a respiratory event (including respiratory depression, respiratory obstruction, cessation of breathing, and/or the like), the continuous sampling engine 362 may increase the sampling rate of a PPG to obtain an increased amount of physiological parameter data (such as PR and SpO2) regarding the event. In some cases, the continuous sampling engine 362 may update the sampling rate according to other aspects of the user's usage. For example, the continuous sampling engine 362 may update the sampling rate when the wearable device obtains data corresponding to the user's activity (e.g., the user begins exercising, sleeping, eating, enters a particular geolocation (such as a gym, hospital, home, school), change in detected stress).

In some instances, the continuous sampling engine 362 may perform intermittent measurement until identifying a likeliness of the physiological event. In response to identifying the likeliness of the physiological event, the continuous sampling engine 362 may perform continuous measurement. In some examples, the continuous sampling engine 362 may obtain physiological parameter data intermittently. For example, the continuous sampling engine 362 may receive the physiological parameter data from the intermittent sampling engine 364. In some cases, the continuous sampling engine 362 may measure the physiological parameter data at a first sampling rate and/or according to the user's instruction. The first sampling rate may be slower than a data freshness standard. In some cases, the continuous sampling engine 362 may identify the likeliness of the physiological event from the intermittent measurement. In response, the continuous sampling engine 362 may continuously measure the physiological parameter data. In some cases, the continuous sampling engine 362 may update the first sampling rate to a second sampling rate. The second sampling rate may be faster than the first sampling rate. Thus, the continuous sampling engine 362 may update from intermittently measuring the physiological data to continuously measuring the physiological data.

The continuous sampling engine 362 may perform threshold-based alert generation to transmit to the alert engine 366. In some instances, the continuous sampling engine 362 may set thresholds for what constitutes a normal physiological parameter range and generate alerts or notifications when the physiological parameter data falls outside these parameters. The continuous sampling engine 362 may compare to standards, such as baseline or objective standards. The baseline may be determined as disclosed herein. The objective standard may include a clinical standard. For example, the continuous sampling engine 362 may compare the physiological parameter data to a model of clinical stability. In some instances, the continuous sampling engine 362 may send an alert signal to the alert engine when the physiological parameter data meets the condition. The continuous sampling engine 362 may perform real-time monitoring. For example, the continuous sampling engine 362 may continuously compare incoming SpO2 readings against predefined thresholds to detect any breaches. In some cases, the continuous sampling engine 362 may perform monitoring from stored data. In some instances, the continuous sampling engine 362 may include conditional statements to continuously compare the latest physiological parameter readings against the thresholds. In some instances, the continuous sampling engine 362 may implement event-driven programming to trigger alerts when thresholds are crossed. In some instances, the continuous sampling engine 362 may include alert triggering logic. For example, logic to trigger alerts when thresholds are breached.

The continuous sampling engine 362 may provide an output to a user interface for data visualization. For example, a user interface that graphically displays physiological parameter trends over time, allowing users to visualize changes in their health. In some instances, the user interface includes a graphical user interface (GUI) design. The GUI may include interactive elements that allow users to zoom in on specific time frames or view detailed information about specific data points. In some cases, the user interface may provide time filtering options, such as options for the user to filter the displayed data by time frame (e.g., second, minutes, hourly, daily, weekly, monthly, yearly) to view short-term fluctuations and long-term trends.

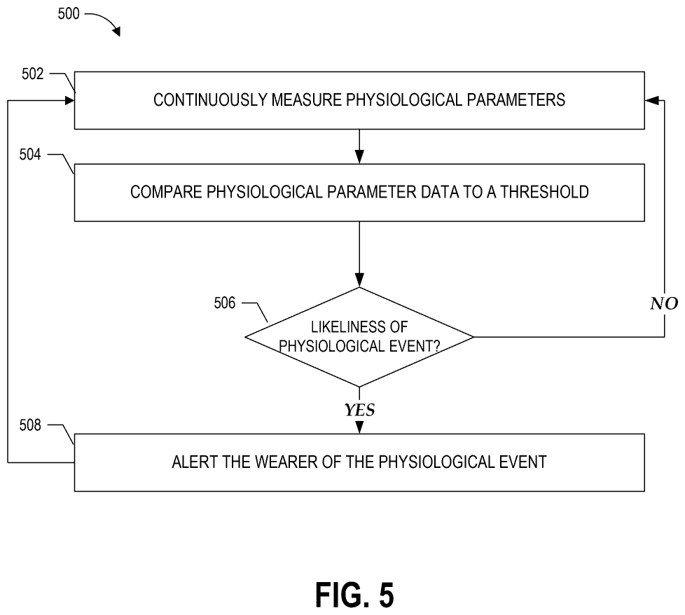

FIG. 5 is one illustrative routine 500 for continuously measure physiological parameters of a user by performing repeated measurements at a series of intervals configured to capture important physiological events. The routine 500 may be carried out, for example, by any of the hardware components as described herein (for example, incorporating the techniques and functionalities as described with the device 10 in FIGS. 1A-2, the hardware processor(s) 902 in FIG. 3A, and the controller 354 in FIG. 3B). For purposes of an example description, the routine 500 is described with reference to the controller 354 (even though any other hardware component as described herein may perform the steps described). The routine 500 begins at block 502, where the controller 354 continuously measures physiological parameters. The controller 354 may measure the physiological parameters by sampling at least one sensor at a sampling rate without interruption while the user is in contact with the wearable device. The at least one sensor may include the sensors as disclosed herein. The physiological parameters may include physiological parameters as disclosed herein. In some examples, the controller 354 may continuously measure the physiological parameters, as described herein (for example, as described in FIGS. 1A-4).

At block 504 the controller 354 compares measurements of the physiological parameters to determine a likelihood of a physiological event. In some examples, the controller 354 may compare the measurements of the physiological parameters to determine the likelihood of the physiological event, as described herein (for example, as described in FIGS. 1A-4).

At block 506 the controller 354 determines a likelihood of the physiological event. In some instances, the controller 354 may estimate the likelihood of the physiological event including quantifiable percentages, confidence scores and/or intervals, qualitative categories, and/or a combination of the foregoing, as disclosed herein. In some instances, the controller 354 may compute a likeliness of a physiological event from various sources of physiological parameter data, as disclosed herein. In some examples, the controller 354 may determine the likelihood of the physiological event (for example, determining a movement condition and/or determining a physiological stability of the user), as described herein (for example, as described in FIGS. 1A-4). When the confidence score indicates the physiological event, the routine 500 proceeds to block 508. When the confidence score indicates the physiological event did not occur, the routine 500 proceeds to block 502.

At block 508 the controller 354 alerts the user of the physiological event. In some instances, the controller 354 may perform threshold-based alert generation, as disclosed herein. In some examples, the controller 354 may take any action in response to the physiological event (for example, selecting a mode of operation), as described herein (for example, as described in FIGS. 1A-4). After alerting the user of the physiological data, the routine 500 proceeds to block 502.

In the above description of FIG. 5, any blocks described can include alternate implementations within the scope of the example embodiments of the present disclosure in which the blocks can be executed out of order from that shown or discussed, including substantially concurrently or in reverse order, depending upon the functionality involved, as would be understood by those skilled in the art. The various elements, features, and processes described herein may be used independently of one another, or may be combined in various ways. All possible combinations and sub-combinations are intended to fall within the scope of this disclosure.

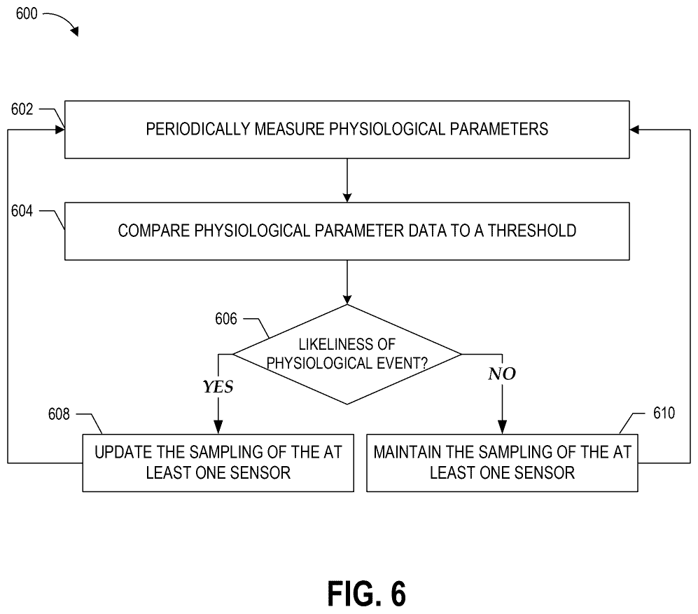

FIG. 6 is one illustrative routine 600 for continuously measuring physiological parameters and updating a sampling rate. The routine 600 may be carried out, for example, by any of the hardware components as described herein (for example, incorporating the techniques and functionalities as described with the device 10 in FIGS. 1A-2, the hardware processor(s) 902 in FIG. 3A, and the controller 354 in FIG. 3B). For purposes of an example description, the routine 500 is described with reference to the controller 354 (even though any other hardware component as described herein may perform the steps described). The routine 600 begins at block 602, where the controller 354 periodically measures physiological parameters by sampling the at least one sensor at a sampling rate. The physiological parameters include the physiological parameters disclosed herein. In some examples, the controller 354 may periodically measure the physiological parameters, as described herein (for example, as described in FIGS. 1A-4).

At block 604 the controller 354 compares measurements of the physiological parameters to a threshold. In some examples, the controller 354 may compare the measurements of the physiological parameters to the threshold, as described herein (for example, as described in FIGS. 1A-4).

At block 606 the controller 354 determines a likelihood of the physiological event. In some instances, the controller 354 may estimate the likelihood of the physiological event including quantifiable percentages, confidence scores and/or intervals, qualitative categories, and/or a combination of the foregoing, as disclosed herein. In some instances, the controller 354 may compute a likeliness of a physiological event from various sources of physiological parameter data, as disclosed herein. In some examples, the controller 354 may determine the likelihood of the physiological event (for example, determining a movement condition and/or determining a physiological stability of the user), as described herein (for example, as described in FIGS. 1A-4). When the confidence score indicates the likelihood of the physiological event is above the predetermined threshold, the routine 600 proceeds to block 608. When the confidence score indicates the physiological event did not occur, the routine 600 proceeds to block 610.

At block 608 the controller 354 updates the sampling rate of the at least one sensor when the likelihood of the physiological event is above a predetermined threshold, as disclosed herein. In some examples, the controller 354 may take any action in response to the physiological event (for example, selecting a mode of operation), as described herein (for example, as described in FIGS. 1A-4).

At block 610 the controller 354 maintains the sampling of the at least one sensor. In some examples, the controller 354 may take any action in response to the absence of the physiological event (for example, selecting a mode of operation), as described herein (for example, as described in FIGS. 1A-4).

In the above description of FIG. 6, any blocks described can include alternate implementations within the scope of the example embodiments of the present disclosure in which the blocks can be executed out of order from that shown or discussed, including substantially concurrently or in reverse order, depending upon the functionality involved, as would be understood by those skilled in the art. The various elements, features, and processes described herein may be used independently of one another, or may be combined in various ways. All possible combinations and sub-combinations are intended to fall within the scope of this disclosure.

### H. Example Alert Engine

The controller 354 may be configured to continuously monitor physiological parameters for determining a physiological event and alert the user of an occurrence of the physiological event. In some examples, the controller 354 may include an alert engine 366 to alert the user when the physiological event occurs. The alert engine 366 may obtain an alert signal from engines disclosed herein (e.g., the continuous sampling engine 362 and the intermittent sampling engine 364). In some cases, the alert engine 366 receives the alert signal in response to the physiological event occurring.

The alert engine 366 may alert the user of the physiological event. The alert engine 366 may include a user interface to communicate data to the user. For example, the user interface may display current SpO2 levels, trends, and alerts on a device or a companion application. In some cases, the alert engine 366 may sync data with a mobile communication device and/or remote server with database for long-term tracking and analysis. The alert engine 366 may notify the user of alerts through the user interface or a companion application. For example, using visual and auditory signals for urgency. The alert engine 366 may include an acknowledgment system, allowing the user to acknowledge alerts and provide feedback on their status. In some instances, the alert engine 366 may reduce alarm fatigue and improve the relevance of notifications.

### I. Additional Embodiments and Terminology

Some inventive aspects of the disclosure are set forth in the following clauses:

Clause 1. A system, comprising: a battery providing operational power; at least one sensor coupled to a wearable device configured to measure physiological parameters of a user; a non-transitory data store storing data collected from the at least one sensor and computer-executable instructions; and a processor in communication with the at least one sensor and the non-transitory data store, wherein the computer-executable instructions, when executed by the processor, configure the processor to: continuously measure the physiological parameters by sampling the at least one sensor at a sampling rate to obtain repeated measurements at series of intervals configured to capture important physiological events; compare measurements of the physiological parameters to a threshold to determine a likelihood of a physiological event; and alert the user when an occurrence of the physiological event is likely.

Clause 2. The system of Clause 1, wherein the likelihood of the physiological event is determined in part by detecting deviations of the physiological parameters from at least one baseline and obtaining a confidence score of the physiological event from a correlation between one or more of the physiological parameters.

Clause 3. The system of Clauses 1 or 2, wherein further computer-executable instructions, when executed by the processor, configure the processor to alert the user when the likelihood of the physiological event is above a predetermined threshold.

Clause 4. The system of one of Clauses 1-3, wherein the physiological event is likely when the measurements indicate at least one of the physiological parameters is outside of a parameter threshold, wherein the parameter threshold includes a low threshold and a high threshold.

Clause 5. The system of Clause 4, wherein the low threshold corresponds to at least one of a user baseline or an objective standard.

Clause 6. The system of Clause 4, wherein the high threshold corresponds to at least one of a user baseline or an objective standard.

Clause 7. The system of Clause 1, wherein the physiological parameters include at least one of blood oxygen, pulse rate (PR), heart rate (HR), skin temperature, core temperature, ambient temperature, movement, body position, respiration rate, heart rate variability (HRV), pulse rate variability (PRV), electrocardiogram (ECG), or acoustic data.

Clause 8. The system of Clause 1, wherein the at least one sensor comprises at least one of: an optical physiological sensor configured to measure the physiological parameters by receiving reflected light emitted by a wearable device from a surface of a user, a microphone configured to record the physiological parameters of the user corresponding to respiration sounds of the user while sleeping, a temperature sensor configured to measure temperature of the user and/or environment surrounding the user, or an accelerometer configured to measure the physiological parameters used to detect a body position of the user while the user is sleeping.

Clause 9. The system of Clause 1, wherein the physiological event is a respiratory event.

Clause 10. The system of Clause 9, wherein the respiratory event comprises one or more of respiratory depression, a respiratory obstruction, or a cessation of breathing.

Clause 11. The system of Clause 1, wherein further computer-executable instructions, when executed by the processor, configure the processor to: continuously measure blood oxygen data and PR data by sampling the at least one sensor at a sampling rate to obtain repeated measurements at series of intervals configured to capture important physiological events.

Clause 12. The system of Clause 1, wherein the sampling rate corresponds to a data freshness standard.

Clause 13. The system of Clause 1, wherein further computer-executable instructions, when executed by the processor, configure the processor to: compare the measurements, including blood oxygen data and PR data, to a threshold to determine a likelihood of a respiratory event corresponding to a respiratory depression, a respiratory obstruction, or a cessation of breathing, wherein the likelihood of the respiratory event is determined in part by detecting deviations of the blood oxygen data and the PR data from baselines and obtaining a confidence score of the respiratory event from a correlation between the blood oxygen data and the PR data.

Clause 14. The system of one of Clauses 1-13, wherein the likelihood of the respiratory event is above the predetermined threshold when the confidence score indicates a correlation between a drop in the blood oxygen data and an increase in the PR data.

Clause 15. The system of one of Clauses 1-14, wherein the likelihood of the respiratory event is above the predetermined threshold when the blood oxygen data is outside of blood oxygen thresholds, including a low blood oxygen threshold and a high blood oxygen threshold.

Clause 16. The system of Clause 15, wherein the low blood oxygen threshold corresponds to at least one of a user baseline or an objective standard.

Clause 17. The system of Clause 15, wherein the high blood oxygen threshold corresponds to at least one of a user baseline or an objective standard.

Clause 18. The system of one of Clauses 1-14, wherein the likelihood of the respiratory event is above the predetermined threshold when the PR data is outside of PR thresholds, including a low PR threshold and a high PR threshold.

Clause 19. The system of Clause 18, wherein the low PR threshold corresponds to at least one of a user baseline or an objective standard.

Clause 20. The system of Clause 18, wherein the high PR threshold corresponds to at least one of a user baseline or an objective standard.

Clause 21. A system, comprising: a battery providing operational power; at least one sensor coupled to a wearable device configured to measure physiological parameters of a user; a non-transitory data store storing data from the at least one sensor and computer-executable instructions; and a processor in communication with the at least one sensor and the non-transitory data store, wherein the computer-executable instructions, when executed by the processor, configure the processor to: periodically measure the physiological parameters by sampling the at least one sensor at a sampling rate; compare measurements of the physiological parameters to a threshold determine a likelihood of a physiological event; and update the sampling rate of the at least one sensor when the physiological event is likely.

Clause 22. The system of Clause 21, wherein the likelihood of the physiological event is determined in part by detecting deviations of the physiological parameters from at least one baseline and obtaining a confidence score of the physiological event from a correlation between one or more of the physiological parameters.

Clause 23. The system of Clause 21, wherein the physiological event is likely when the measurements indicate at least one of the physiological parameters is outside of a parameter threshold, including a low threshold and a high threshold.

Clause 24. The system of Clause 23, wherein the low threshold corresponds to at least one of a user baseline or an objective standard.

Clause 25. The system of Clause 23, wherein the high threshold corresponds to at least one of a user baseline or an objective standard.

Clause 26. The system of one of Clauses 21-25, wherein the physiological parameters include at least one of blood oxygen, pulse rate (PR), heart rate (HR), skin temperature, core temperature, ambient temperature, movement, body position, respiration rate, heart rate variability (HRV), pulse rate variability (PRV), electrocardiogram (ECG), or acoustic data.

Clause 27. The system of one of Clauses 21-26, wherein the at least one sensor comprises at least one of: an optical physiological sensor configured to measure the physiological parameters by receiving reflected light emitted by a wearable device from a surface of a user, a microphone configured to record the physiological parameters of the user corresponding to respiration sounds of the user while sleeping, a temperature sensor configured to measure temperature of the user and/or environment surrounding the user, or an accelerometer configured to measure the physiological parameters used to detect a body position of the user while the user is sleeping.

Clause 28. The system of one of Clauses 21-27, wherein the physiological event is a respiratory event.

Clause 29. The system of Clause 28, wherein the respiratory event comprises at least one of respiratory depression, a respiratory obstruction, or a cessation of breathing.

Clause 30. The system of one of Clauses 21-29, wherein further computer-executable instructions, when executed by the processor, configure the processor to: measure body position data by sampling the accelerometer at a second sampling rate.

Clause 31. The system of Clause 21, wherein further computer-executable instructions, when executed by the processor, configure the processor to: compare the measurements, including body position data of the user, to blood oxygen data to determine a likelihood of a respiratory event corresponding to a respiratory depression, a respiratory obstruction, or a cessation of breathing, wherein the likelihood of the respiratory event is determined in part by detecting a deviation of the blood oxygen data from a baseline and obtaining a confidence score of the respiratory event from a correlation between the blood oxygen data and the body position data.

Clause 32. The system of Clause 21, wherein further computer-executable instructions, when executed by the processor, configure the processor to: update the sampling rate of the at least one sensor to a new sampling rate to measure blood oxygen data when the likelihood of a respiratory event is above a predetermined threshold, wherein the new sampling rate is greater than the sampling rate.

Clause 33. The system of one of Clauses 21-32, wherein the likelihood of the respiratory event is above the predetermined threshold when the confidence score indicates a correlation between a drop in the blood oxygen data and the body position data of the user in a position increasing a risk of the respiratory event.

Clause 34. The system of one of Clauses 21-32, wherein the likelihood of the respiratory event is above the predetermined threshold when the blood oxygen data is outside of blood oxygen thresholds, including a low blood oxygen threshold and a high blood oxygen threshold.

Clause 35. The system of Clause 34, wherein the low blood oxygen threshold corresponds to at least one of a user baseline or an objective standard.

Clause 36. The system of Clause 34, wherein the high blood oxygen threshold corresponds to at least one of a user baseline or an objective standard.

Clause 37. A method of operating a wearable system for continuously monitoring physiological data of a wearer over an extended period using a set of physiological sensors by dynamically scheduling power consumption of the set of physiological sensors based at least in part on the physiological data, the method comprising: determining a movement condition of the wearable system based at least in part on data from at least one motion sensor, the movement condition comprising elevated movement or reduced movement; determining a physiological stability of the wearer based at least in part on at least one physiological signal, the physiological stability comprising a stable condition or an unstable condition; selecting a mode of operating a plurality of LEDs based at least in part on the movement condition and the physiological stability of the wearer, wherein a first mode of operation is associated with elevated movement or unstable physiological condition, wherein the first mode of operation comprises a first power management condition comprising permitting the plurality of LEDs to emit light, wherein a second mode of operation is associated with reduced movement and stable physiological condition, wherein the second mode of operation comprises a second power management condition comprising permitting to emit light a first set of the plurality of LEDS that are configured to emit light within a first wavelength range and disallowing from emitting light a second set of the plurality of LEDs that are configured to emit light in a second wavelength range, wherein the first set is different from the second set; and adjusting operation of the plurality of LEDs based on the selected mode.

Clause 38. The method of clause 37, wherein the first power management condition comprises permitting all LEDs of the plurality of LEDs to emit light.

Clause 39. The method of clause 37, wherein the first power management condition comprises permitting to emit light a first set of the plurality of LEDs that are configured to emit light within the first wavelength range and disallowing from emitting light a second set of the plurality of LEDs that are configured to emit light within the second wavelength range, wherein the first set is different from the second set, and wherein disallowing the second set from emitting light is based on hardware considerations or physiological considerations.

Clause 40. The method of clause 37, the method further comprising, when operating in the second mode, maintaining a buffer of physiological data associated with light within the second wavelength range after attenuation by tissue of the wearer.

Clause 41. The method of clause 37, wherein the first wavelength range corresponds to visible light.

Clause 42. The method of clause 37, wherein the second wavelength range corresponds to visible light or infrared wavelengths.

Clause 43. The method of clause 37, wherein the at least one physiological signal is associated with the light after attenuation by tissue corresponding to a wrist or a finger of the wearer.

Clause 44. The method of clause 37, wherein the at least one physiological signal is associated with light within the first wavelength range after attenuation by tissue of the wearer.

Clause 45. The method of clause 37, further comprising a third mode of operation that is associated with reduced movement and stable physiological condition, wherein the third mode of operation comprises a third power management condition comprising disallowing from emitting light the plurality of LEDs.

Clause 46. The method of clause 37, wherein adjusting operation of the plurality of LEDs comprises increasing a period of the first mode or decreasing a period of the first mode, or increasing a period of the second mode or decreasing a period of the second mode.

Clause 47. The method of clause 37, wherein the first power management condition comprises turning on additional motion sensors configured to detect motion of the wearable system.

Clause 48. The method of clause 45, wherein the third power management condition comprises turning off the at least one motion sensor.

Clause 49. The method of clause 37, wherein determining whether the wearer has a stable physiological condition or an unstable physiological condition comprises determining whether a physiological threshold condition is satisfied.

Clause 50. The method of clause 49, wherein the physiological threshold condition is based in part on a history of physiological data received from one or more detectors.

Clause 51. The method of clause 50, wherein determining whether the physiological threshold condition is satisfied comprises determining of whether a value, a change, a rate of change, or a change in the rate of change of the physiological data satisfies the physiological threshold condition.

Clause 52. The method of clause 37, wherein determining whether the movement condition is elevated or reduced comprises determining whether a movement threshold condition is satisfied.

Clause 53. The method of clause 52, wherein the movement threshold condition is based in part on a history of movement data received from the at least one motion sensor.

Clause 54. The method of clause 53, wherein determining whether the movement threshold condition is satisfied comprises determining whether a value, a change, a rate of change, or a change in the rate of change of the movement data satisfies the movement threshold condition.

Clause 55. The method of clause 37, wherein the at least one motion sensor is an accelerometer or gyroscope.

Clause 56. A system configured to implement a method as described in any of Clauses 37-55.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain aspects, the terms “generally parallel” and “substantially parallel” refer to a value, amount, or characteristic that departs from exactly parallel by less than or equal to 10 degrees, 5 degrees, 3 degrees, or 1 degree. As another example, in certain aspects, the terms “generally perpendicular” and “substantially perpendicular” refer to a value, amount, or characteristic that departs from exactly perpendicular by less than or equal to 10 degrees, 5 degrees, 3 degrees, or 1 degree.

Many other variations than those described herein will be apparent from this disclosure. For example, certain acts, events, or functions of any of the algorithms described herein can be performed in a different sequence, can be added, merged, or left out altogether (for example, not all described acts or events are necessary for the practice of the algorithms). Moreover, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

It is to be understood that not necessarily all such advantages can be achieved in accordance with any particular example of the examples disclosed herein. Thus, the examples disclosed herein can be embodied or carried out in a manner that achieves or optimizes one advantage or group of advantages as taught herein without necessarily achieving other advantages as may be taught or suggested herein.

The various illustrative logical blocks, modules, and algorithm steps described in connection with the examples disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the examples disclosed herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry or digital logic circuitry configured to process computer-executable instructions. In another example, a processor can include an FPGA or other programmable device that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. A computing environment can include any type of computer system, including, but not limited to, a computer system based on a microprocessor, a mainframe computer, a digital signal processor, a portable computing device, a device controller, or a computational engine within an appliance, to name a few.

The steps of a method, process, or algorithm described in connection with the examples disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC.

The apparatuses and methods described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

The term “substantially” when used in conjunction with the term “real-time” forms a phrase that will be readily understood by a person of ordinary skill in the art. For example, it is readily understood that such language will include speeds in which no or little delay occurs.

Conditional language used herein, such as, among others, “can,” “might,” “may,” “for example,” and the like, unless specifically stated otherwise, or otherwise understood within the context as used, is generally intended to convey that certain examples include, while other examples do not include, certain features, elements and/or states. Thus, such conditional language is not generally intended to imply that features, elements and/or states are in any way required for one or more examples or that one or more examples necessarily include logic for deciding, with or without author input or prompting, whether these features, elements and/or states are included or are to be performed in any particular example. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Disjunctive language such as the phrase “at least one of X, Y, or Z,” unless specifically stated otherwise, is otherwise understood with the context as used in general to present that an item, term, etc., may be either X, Y, or Z, or any combination thereof (for example, X, Y, and/or Z). Thus, such disjunctive language is not generally intended to, and should not, imply that certain examples require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C. Unless otherwise explicitly stated, the terms “set” and “collection” should generally be interpreted to include one or more described items throughout this application. Accordingly, phrases such as “a set of devices configured to” or “a collection of devices configured to” are intended to include one or more recited devices. Such one or more recited devices can also be collectively configured to carry out the stated recitations. For example, “a set of servers configured to carry out recitations A, B and C” can include a first server configured to carry out recitation A working in conjunction with a second server configured to carry out recitations B and C.

While the above detailed description has shown, described, and pointed out novel features as applied to various examples, it will be understood that various omissions, substitutions, and changes in the form and details of the devices or algorithms illustrated can be made without departing from the spirit of the disclosure. As will be recognized, the inventions described herein can be embodied within a form that does not provide all of the features and benefits set forth herein, as some features can be used or practiced separately from others.

Additionally, all publications, patents, and patent applications mentioned in this specification are herein incorporated by reference to the same extent as if each individual publication, patent, or patent application was specifically and individually indicated to be incorporated by reference.

What is claimed is:

1. A system, comprising:
at least one sensor coupled to a wearable device, comprising an optical physiological sensor configured to measure physiological parameters by receiving reflected light emitted by a wearable device from a surface of a user;
a non-transitory data store storing data collected from the at least one sensor and computer-executable instructions; and
a processor in communication with the at least one sensor and the non-transitory data store, wherein the computer-executable instructions, when executed by the processor, configure the processor to:
continuously obtain measurements of blood oxygen data and PR data by sampling the at least one sensor at a sampling rate to obtain repeated measurements at series of intervals configured to capture important physiological events;
compare the measurements, including blood oxygen data and PR data, to a threshold to determine a likelihood of a respiratory event corresponding to a respiratory depression, a respiratory obstruction, or a cessation of breathing, wherein the likelihood of the respiratory event is determined in part by detecting deviations of the blood oxygen data and the PR data from baselines and obtaining a confidence score of the respiratory event from a correlation between the blood oxygen data and the PR data; and
alert the user when an occurrence of the physiological event is likely.

2. The system of claim 1, wherein the physiological parameters include at least one of blood oxygen, pulse rate (PR), heart rate (HR), skin temperature, core temperature, ambient temperature, movement, body position, respiration rate, heart rate variability (HRV), pulse rate variability (PRV), electrocardiogram (ECG), or acoustic data.

3. The system of claim 1, wherein the at least one sensor comprises at least one of: a microphone configured to record the physiological parameters of the user corresponding to respiration sounds of the user while sleeping, a temperature sensor configured to measure temperature of the user and/or environment surrounding the user, or an accelerometer configured to measure the physiological parameters used to detect a body position of the user while the user is sleeping.

4. The system of claim 1, wherein the physiological event is a respiratory event, wherein the respiratory event comprises one or more of respiratory depression, a respiratory obstruction, or a cessation of breathing.

5. The system of claim 1, wherein the sampling rate corresponds to a data freshness standard.

6. The system of claim 1, wherein the likelihood of the respiratory event is above the threshold when the confidence score indicates a correlation between a drop in the blood oxygen data and an increase in the PR data.

7. The system of claim 1, wherein the likelihood of the respiratory event is above the threshold when the blood oxygen data is outside of blood oxygen thresholds, including a low blood oxygen threshold and a high blood oxygen threshold.

8. The system of claim 7, wherein the low blood oxygen threshold corresponds to at least one of a user baseline or an objective standard.

9. The system of claim 7, wherein the high blood oxygen threshold corresponds to at least one of a user baseline or an objective standard.

10. The system of claim 1, wherein the likelihood of the respiratory event is above the threshold when the PR data is outside of PR thresholds, including a low PR threshold and a high PR threshold.

11. The system of claim 10, wherein the low PR threshold corresponds to at least one of a user baseline or an objective standard.

12. The system of claim 10, wherein the high PR threshold corresponds to at least one of a user baseline or an objective standard.

13. A system, comprising:
at least one sensor coupled to a wearable device, comprising an optical physiological sensor configured to measure physiological parameters by receiving reflected light emitted by a wearable device from a surface of a user;
a non-transitory data store storing data from the at least one sensor and computer-executable instructions; and
a processor in communication with the at least one sensor and the non-transitory data store, wherein the computer-executable instructions, when executed by the processor, configure the processor to:
periodically obtain measurements of the physiological parameters by sampling the at least one sensor at a sampling rate;
compare the measurements, including body position data of the user, to blood oxygen data to determine a likelihood of a respiratory event corresponding to a respiratory depression, a respiratory obstruction, or a cessation of breathing, wherein the likelihood of the respiratory event is determined in part by detecting a deviation of the blood oxygen data from a baseline and obtaining a confidence score of the respiratory event from a correlation between the blood oxygen data and the body position data; and
update the sampling rate of the at least one sensor to a new sampling rate to measure blood oxygen data when the likelihood of a respiratory event is above a predetermined threshold, wherein the new sampling rate is greater than the sampling rate.

14. The system of claim 13, wherein the physiological parameters include at least one of blood oxygen, pulse rate (PR), heart rate (HR), skin temperature, core temperature, ambient temperature, movement, body position, respiration rate, heart rate variability (HRV), pulse rate variability (PRV), electrocardiogram (ECG), or acoustic data.

15. The system of claim 13, wherein the at least one sensor comprises at least one of: a microphone configured to record the physiological parameters of the user corresponding to respiration sounds of the user while sleeping, a temperature sensor configured to measure temperature of the user and/or environment surrounding the user, or an accelerometer configured to measure the physiological parameters used to detect a body position of the user while the user is sleeping.

16. The system of claim 13, wherein the physiological event is a respiratory event, wherein the respiratory event comprises at least one of respiratory depression, a respiratory obstruction, or a cessation of breathing.

17. The system of claim 13, wherein the likelihood of the respiratory event is above the predetermined threshold when the confidence score indicates a correlation between a drop in the blood oxygen data and the body position data of the user in a position increasing a risk of the respiratory event.

18. The system of claim 13, wherein the likelihood of the respiratory event is above the predetermined threshold when the blood oxygen data is outside of blood oxygen thresholds, including a low blood oxygen threshold and a high blood oxygen threshold.

19. The system of claim 18, wherein the low blood oxygen threshold corresponds to at least one of a user baseline or an objective standard.

20. The system of claim 18, wherein the high blood oxygen threshold corresponds to at least one of a user baseline or an objective standard.

21. A method of operating a wearable system for continuously monitoring physiological data of a wearer over an extended period using a set of physiological sensors by dynamically scheduling power consumption of the set of physiological sensors based at least in part on the physiological data, the method comprising:
determining a movement condition of the wearable system based at least in part on data from at least one motion sensor, the movement condition comprising elevated movement or reduced movement;
determining a physiological stability of the wearer based at least in part on at least one physiological signal, the physiological stability comprising a stable condition or an unstable condition;
selecting a mode of operating a plurality of LEDs based at least in part on the movement condition and the physiological stability of the wearer,
wherein a first mode of operation is associated with elevated movement or unstable physiological condition, wherein the first mode of operation comprises a first power management condition comprising permitting the plurality of LEDs to emit light,
wherein a second mode of operation is associated with reduced movement and stable physiological condition, wherein the second mode of operation comprises a second power management condition comprising permitting to emit light a first set of the plurality of LEDS that are configured to emit light within a first wavelength range and disallowing from emitting light a second set of the plurality of LEDs that are configured to emit light in a second wavelength range, wherein the first set is different from the second set; and
adjusting operation of the plurality of LEDs based on the selected mode.
